SEQUENCE LISTING (14 sequences)

SEQ ID NO: 1 - DNA, 23 bp, Artificial Sequence
  synthetic oligo
catcgcctggatccccgggaacg

SEQ ID NO: 2 - DNA, 27 bp, Artificial Sequence
  synthetic oligo
gacgcaaaaagcttttgatgacgcgcc

SEQ ID NO: 3 - DNA, 31 bp, Artificial Sequence
  synthetic oligo
gaggatccacaggaagcttgatgcgttttag

SEQ ID NO: 4 - DNA, 32 bp, Artificial Sequence
  synthetic oligo
cgcaagatttagggatccttcagaatacgacc

SEQ ID NO: 5 - DNA, 19 bp, Artificial Sequence
  synthetic oligo
gcgagaatacggagaattg

SEQ ID NO: 6 - DNA, 17 bp, Artificial Sequence
  synthetic oligo
gaggggaaaaattgcag

SEQ ID NO: 7 - DNA, 20 bp, Artificial Sequence
  synthetic oligo
acgtttcagtttgctcatgg

SEQ ID NO: 8 - DNA, 20 bp, Artificial Sequence
  synthetic oligo
ccggcctttattcacattct

SEQ ID NO: 9 - DNA, 18 bp, Artificial Sequence
  synthetic oligo
cagctgcaggtagcttcg

SEQ ID NO: 10 - DNA, 20 bp, Artificial Sequence
  synthetic oligo
tatgctgatgtggtcagggt

SEQ ID NO: 11 - DNA, 20 bp, Artificial Sequence
  synthetic oligo
attcttacccgctcatctgg

SEQ ID NO: 12 - DNA, 20 bp, Artificial Sequence
  synthetic oligo
gtggcaacaatttccacttg

SEQ ID NO: 13 - DNA, 19 bp, Artificial Sequence
  synthetic oligo
aacgtaccttcgtcagcga

SEQ ID NO: 14 - DNA, 20 bp, Artificial Sequence
  synthetic oligo
gtggcgttgattgtagtggt

(19) United States
(12) Patent Application Publication
(10) Pub. No.: US 2009/0208461 A1
(43) Pub. Date: Aug. 20, 2009

(54) RECOMBINANT BACTERIA WITH E.COLI HEMOLYSIN SECRETION SYSTEM AND INCREASED EXPRESSION AND/OR SECRETION OF HLYA, PROCESS OF MANUFACTURING AND USES THEREOF

(75) Inventors: Christian HOTZ, Muenchen (DE); Ivaylo Gentschev, Kist (DE); Ulf Rapp, Wuerzburg (DE); Werner Goebel, Muenchen (DE); Joachim Fensterle, Hoechberg (DE)

Correspondence Address: OBLON, SPIVAK, MCCLELLAND MAIER & NEUSTADT, P.C., 1940 DUKE STREET, ALEXANDRIA, VA, 22314, US

(73) Assignee: ZENTARIS GmbH, Frankfurt am Main (DE)

(21) Appl. No.: 12/365,944

(22) Filed: Feb. 5, 2009

Related U.S. Application Data
(60) Provisional application No. 61/026,158, filed on Feb. 5, 2008.

(30) Foreign Application Priority Data
Feb. 5, 2008 (EP) 08101280.9

(51) Int. Cl. A61K 35/74; C12N 1/21; C12N 15/87

(52) U.S. Cl. 424/93.2; 435/252.3; 435/252.33; 435/252.34; 435/471

(57) ABSTRACT

The invention relates to a recombinant bacterium with E. coli hemolysin secretion system and increased expression and/or increased secretion of full length or partial HlyA.

### CROSS-REFERENCE TO RELATED APPLICATIONS

This application claims the benefit of EP 08101280.9 filed Feb. 5, 2008 and U.S. 61/026,158 filed Feb. 5, 2008, each of which is incorporated herein by reference.

### DESCRIPTION OF THE INVENTION

1. Field of the Invention

The invention relates to recombinant bacteria with E. coli hemolysin secretion system and increased expression and/or increased secretion of full length or partial HlyA and a process of manufacturing thereof. These recombinant bacteria can be used as medicaments, in particular for the treatment of various tumors.

2. Background of the Invention

The licensed typhoid vaccine strain Ty21a is an attenuated mutant strain of S. typhi Ty2. The attenuation of the vaccine strain is due to an irreversible genetic defect, achieved by multiple mutations induced by chemical mutagenesis [1]. These mutations led to a strain which is sensitive to galactose, (mutation in the galE gene), auxotrophic for amino acids isoleucine and valine (mutation of ilvD genes), lacks the polysaccharide capsule (mutation in via) and has a reduced stress resistance (mutationin rpoS) [2-5]. The multiple mutations of Ty21a collectively render it genetically stable. Reversion to virulence has been observed neither in vitro nor in vivo. Due to the vast experience with Ty21a, this strain is an obvious candidate as a carrier for heterologous antigens. Two recent clinical trials assessed Ty21a as a carrier for antigens of Helicobacter pylori [6, 7]. In these trials, experimental formulations were assessed; a Ty21a strain expressing urease A and B subunits of H. pylori was grown in plant-based Luria Bertani broth and administered orally after mixing either freshly harvested cultures or frozen aliquots with bicarbonate buffer. The strain was found to be safe and immunogenic in both studies. The bacteria elicited humoral and cellular immune responses against S. typhi and cellular immunity against urease. Interestingly, previous immunization with Ty21a did not exhibit any negative impact on the urease-specific immune responses [7]. Nevertheless, only 56% of vaccines exhibited cellular immunity against urease, which may be due to the fact that the heterologous antigen was expressed in a cytoplasmic fashion. It was recently shown in numerous preclinical studies that secretion or surface-display of heterologous antigens by salmonellae leads to superior immunogenicity in comparison to cytoplasmic expression [8,9]. One of the most promising approaches in this direction is the use of the E. coli alpha-hemolysin (HlyA) secretion system for antigen delivery [10]. This transport machinery is the prototype of type I secretion systems (T1SS) and consists of three different components, namely HlyB, HlyD and TolC [11]. The HlyA carries at its C-terminus a secretion signal of about 50-60 amino acids in length (HlyAₛ), which is recognized by the HlyB/HlyD/TolC-translocator, leading to direct secretion of the entire protein into the extracellular medium. The fusion of the HlyAₛ to the C-terminus of heterologous antigens leads to efficient secretion of such proteins by the recombinant bacteria. The system is also fully functional in a wide range of gram-negative bacteria, including several experimentally attenuated Salmonella strains [10, 12, 13].

### BRIEF DESCRIPTION OF THE DRAWINGS

FIG. 1 Identification of hemolysin by Western blot (WB): Cultures of S. typhi Ty21a (lanes 1 and 2), S. typhi Ty21a/pANN202-812 (lanes 3 and 4), S. typhimurium SL7207/pANN202-812 (lanes 5 and 6) and S. dublin SL5928/pANN202-812 (lanes 7 and 8) were analyzed. Cellular proteins of 0.05 ml bacterial culture were loaded in lanes 1, 3, 5 and 7; supernatant proteins precipitated from 2.5 ml of the bacterial culture were loaded in lanes 2, 4, 6 and 8. The immunoblot was developed with polyclonal anti HlyAs-antibodies [12, 16].

FIG. 2 (WB) Complementation Ty21a/pANN202-812 with pRpoS: Cultures of Ty21a/pANN202-812 (lanes 1-4), rpoSTy21a/paNN202-812 (lanes 5-8) and Ty21a alone (lane 9). Cellular proteins of 0.12 ml were loaded on the gel (lanes 1, 3, 5 and 7). Supernatants precipitated from 2.5 ml culture were applied in lanes 2, 4, 6, 8 and 9). Samples were taken in the logarithmic (lanes 1, 2, 5 and 6) or in the stationary phase (3, 4, 7, 8 and 9). The immunoblot was developed with polyclonal anti HlyAs antibodies [12, 16].

FIG. 3 Effects of rfaH on transcription, expression and secretion. WB (A), semi-quantitative RT-PCR (B, C): A: Cultures of Ty21a/pANN202-812 (lanes 1-4), rfaHTy21a/paNN202-812 (lanes 5-8). Cellular proteins of 0.12 ml were loaded on the gel (lanes 1, 3, 5 and 7). Supernatants precipitated from 2.5 ml culture were applied in lanes 2, 4, 6, 8 and 9). Samples were taken in the logarithmic (lanes 1, 2, 5 and 6) or in the stationary phase (3, 4, 7, and 8). The immunoblot was developed with polyclonal anti HlyAs antibodies [12, 16]. B, C: RNA was isolated from cultures of Ty21a/pANN202-812 and rfaHTy21a/paNN202-812 grown to the early logarithmic (B) or stationary phase (C) and reverse transcribed into cDNA. Indicated genes were analysed in a Rotor-gene Real-time PCR. The relative changes in gene expression between different strains were calculated after normalization with the cat gene as internal control. Significances in changes of regulation were calculated using Students T-Test. *=P-value<0.05; **=P-value<0.01; =P-value value<0.001

FIG. 4 Invasion and intracellular survival of Ty21a, rpoSTy21a (A) and rfaHTy21a (B) in RAW 264.7 macrophages: Cells were infected at a multiplicity of infection of 100 and lysed after two and four hours post infection. CFU was determined by plating serial dilutions on LB agar plates. Relative CFU was calculated by comparing CFUs of different strains with Ty21a at 2 h post infection (p.i.). Significances in relative CFUs were determined using Students T-Test. *=P-value<0.05; **=P-value<0.01

FIG. 5. HlyA (A) and LPS-specific (B) serum antibody responses of mice immunized with rpoSTy21a/pANN202-812, rfaHTy21a/pANN202-812, Ty21a/pANN202-812, Ty21a (control) and naïve mice, determined by HlyA or LPS-specific ELISA with anti IgG (A) and anti IgG+IgM (B) detection antibodies. Data were analyzed by 1-way-Anova followed by Newman-Keuls multiple comparison test. *=P-value<0.05; **=P-value<0.01

### DESCRIPTION OF THE INVENTION

The present invention has the object to provide novel tumor vaccines by means of which a more efficient tumor therapy can be achieved.

The object of the present invention has been surprisingly solved in one aspect by providing a recombinant bacterium which comprises at least one nucleotide sequence coding for the E. coli hemolysin secretion system, wherein the at least one nucleotide sequence comprises full length or partial HlyA, HlyB and HlyD gene sequences under control of the hly-specific promoter or a not hly-specific bacterial promoter, and which further comprises at least one nucleotide sequence coding for a protein that effects an increased expression and/or increased secretion of full length or partial HlyA compared to normal/wild-type HlyA expression and/or secretion.

In a preferred embodiment, the recombinant bacterium according to above aspects and embodiments further possesses a deleted or inactivated rpoS gene.

In another preferred embodiment, the further comprised at least one nucleotide sequence comprises rfaH and/or rpoN gene and is integrated into the bacterial chromosome or, preferably, is located on a plasmid.

In another preferred embodiment, the recombinant bacterium according to above aspects and embodiments is attenuated.

In yet another preferred embodiment, the attenuation is caused by deletion or inactivation of at least one gene selected from the group consisting of: aroA, aro, asd, gal, pur, cya, crp, phoP/Q, omp.

In yet another preferred embodiment, the attenuation results in an auxotrophic bacterium.

In a further preferred embodiment, the recombinant bacterium according to above aspects and embodiments is selected from the group consisting of: gram-negative bacterium, gram-positive bacterium.

In a further preferred embodiment, the recombinant bacterium according to above aspects and embodiments is selected from the group consisting of: Shigella spp., Salmonella spp., Listeria spp., Escherichia spp., Mycobacterium spp., Yersinia spp., Vibrio spp., Pseudomonas spp.

In a further preferred embodiment, the recombinant bacterium according to above aspects and embodiments is selected from the group consisting of: Shigella flexneri, Salmonella typhimurium, Mycobacterium bovis BCG, Listeria monocytogenes, Salmonella typhi, Yersinia enterocolitica, Vibrio cholerae, Escherichia coli and preferably is selected from the group consisting of: Salmonella typhi Ty2, Salmonella typhi Ty21a.

In a further preferred embodiment, the recombinant bacterium according to above aspects and embodiments further comprises at least one nucleotide sequence coding for at least one complete or partial antigen of at least one wild-type or mutated protein and at least one nucleotide sequence coding for at least one protein toxin and/or at least one protein toxin subunit.

In a further preferred embodiment, the at least one complete or partial antigen of at least one wild-type or mutated protein according to component (I) is selected from the group consisting of the following wild-type proteins and their known mutants: receptor; extracellular, transmembranic or intracellular part of a receptor; adhesion molecule; extracellular, transmembranic or intracellular part of an adhesion molecule; signal-transducing protein; cell-cycle protein; transcription factor; differentiation protein; embryonic protein; viral protein; allergen; protein of microbial pathogen; protein of eukaryotic pathogen; cancer testis antigen protein; tumor antigen protein; and/or tissue-cell specific protein, wherein the tissue cell is selected from the group consisting of: glandula thyroidea, glandula mammaria, glandula salivaria, nodus lymphoideus, glandula mammaria, tunica mucosa gastris, kidney, ovarium, prostate, cervix, tunica serosa vesicae urinariae and nevus.

As for the mutated protein, the mutation may have been oncogenic and may have caused a loss or a gain of its original cellular functions.

Such antigens perform in the cell the control of the cell growth and of the cell division and are presented on the cell membrane of normal cells, for instance by the MHC class I molecule. In tumor cells, these antigens are frequently over-expressed or specifically mutated. Such mutations can have function limitations of oncogene suppressors or the activation of proto-oncogenes to oncogenes as a consequence and can be involved alone or commonly with over-expressions in the tumor growth. Such cell antigens are presented on the membrane of tumor cells and thus represent antigens on tumor cells, without however causing an immune reaction affecting the tumor disease of the patient. Rapp (U.S. Pat. No. 5,156,841) has already described the use of oncoproteins, i.e. expression products of the oncogenes, as an immunogen for tumor vaccines.

Examples for antigens and their (oncogenic) mutations according to the invention are i) receptors, such as Her-2/neu, androgen receptor, estrogen receptor, lactoferrin receptor, midkine receptor, EGF receptor, ERBB2, ERBB4, TRAIL receptor, FAS, TNFalpha receptor, TGF-beta receptor; ii) signal-transducing proteins, such as c-Raf (Raf-1), A-Raf, B-Raf, B-Raf V599E, B-Raf V600E, B-Raf KD, B-Raf V600E kinase domain, B-Raf V600E KD, B-Raf V600E kinase domain KD, B-Raf kinase domain, B-Raf kinase domain KD, Ras, Bcl-2, Bcl-X, Bcl-W, Bfl-1, Brag-1, Mcl-1, A1, Bax, BAD, Bak, Bcl-Xs, Bid, Bik, Hrk, Bcr/abl, Myb, C-Met, IAP1, IAO2, XIAP, ML-IAP LIVIN, survivin, APAF-1; iii) proteins of the cell cycle control, such as cyclin D(1-3), cyclin E, cyclin A, cyclin B, cyclin H, Cdk-1, Cdk-2, Cdk-4, Cdk-6, Cdk-7, Cdc25C, p16, p15, p21, p27, p18, pRb, p107, p130, E2F(1-5), GAAD45, MDM2, PCNA, ARF, PTEN, APC, BRCA, p53 and homologues; iv) transcription factors, such as C-Myc, NFkB, c-Jun, ATF-2, Spl; v) embryonic proteins, such as carcinoembryonic antigen, alpha-fetoprotein, MAGE, MAGE-1, MAGE-3, NY-ESO-1, PSCA; vi) differentiation antigens, such as MART, Gp100, tyrosinase, GRP, TCF-4, basic myelin, alpha-lactalbumin, GFAP, prostate specific antigen (PSA), fibrillary acid protein, tyrosinase, EGR-1, MUC1; vii) viral antigens, such as of the following viruses: HIV, HPV, HCV, HPV, EBV, CMV, HSV, influenza virus, influenza virus type A, influenza virus type A (H5N1) and (H3N2), influenza virus type B, influenza virus type C; hemagglutinins, hemagglutinin H1, hemagglutinin H5, hemagglutinin H7, hemagglutinin HA1 (preferably from Influenza A virus (A/Thailand/1 (KAN-1)2004(H₅N1), hemagglutinin HA12 (preferably from Influenza A virus (A/Thailand/1 (KAN-1)2004(H5N1), hemagglutinin HA12C (preferably from Influenza A virus (A/Thailand/1 (KAN-1)2004(H5N1), neuramidase, microbial antigens: p60, LLO, urease etc. Antigens of eukaryotic pathogens: CSP (malaria), calflagin (trypanosoma), CPB (Leishmania major) etc.

In a yet further preferred embodiment, the at least one complete or partial antigen of at least one wild-type or mutated protein according to component (I) is selected from the group consisting of the following wild-type proteins and their known mutants: Her-2/neu, androgen receptor, estrogen receptor, midkine receptor, EGF receptor, ERBB2, ERBB4, TRAIL receptor, FAS, TNFalpha receptor, TGF-beta receptor, lactoferrin receptor, basic myelin, alpha-lactalbumin, GFAP, fibrillary acid protein, tyrosinase, EGR-1, MUC1, c-Raf (Raf-1), A-Raf, B-Raf, B-Raf V599E, B-Raf V600E, B-Raf KD, B-Raf V600E kinase domain, B-Raf V600E KD, B-Raf V600E kinase domain KD, B-Raf kinase domain, B-Raf kinase domain KD, N-Ras, K-Ras, H-Ras, Bcl-2, Bcl-X, Bcl-W, Bfl-1, Brag-1, Mcl-1, A1, Bax, BAD, Bak, Bcl-Xs, Bid, Bik, Hrk, Bcr/abl, Myb, C-Met, IAP1, IAO2, XIAP, ML-IAP LIVIN, survivin, APAF-1, cyclin D(1-3), cyclin E, cyclin A, cyclin B, cyclin H, Cdk-1, Cdk-2, Cdk-4, Cdk-6, Cdk-7, Cdc25C, p16, p15, p21, p27, p18, pRb, p107, p130, E2F(1-5), GAAD45, MDM2, PCNA, ARF, PTEN, APC, BRCA, Akt, PI3K, mTOR, p53 and homologues, C-Myc, NFkB, c-Jun, ATF-2, Sp1, prostate specific antigen (PSA), carcinoembryonic antigen, alpha-fetoprotein, PAP; PSMA; STEAP; MAGE, MAGE-1, MAGE-3, NY-ESO-1, PSCA, MART, Gp100, tyrosinase, GRP, TCF-4, viral antigens of the viruses HIV, HPV, HCV, HPV, EBV, CMV, HSV, influenza virus, influenza virus type A, influenza virus type A (H5N1) and (H3N2), influenza virus type B, influenza virus type C; hemagglutinins, hemagglutinin H1, hemagglutinin H5, hemagglutinin H7, hemagglutinin HA1 (preferably from Influenza A virus (A/Thailand/1 (KAN-1)2004(H5N1), hemagglutinin HA12 (preferably from Influenza A virus (A/Thailand/1 (KAN-1)2004(H5N1), hemagglutinin HA12C (preferably from Influenza A virus (A/Thailand/1 (KAN-1)2004(H5N1), neuramidase, p60, LLO, urease, CSP, calflagin and/or CPB or wherein the at least one complete or partial antigen of at least one wild-type or mutated protein according to component (I) is selected from the group of kinases consisting of the following wild-type proteins and their known mutants (accession numbers in parantheses): MK1 (NM 014911), AATK (NM 004920), ABL1 (NM 005157), ABL2 (NM 005158), ACK1 (NM 005781), ACVR1 (NM 001105), ACVR1B (NM 020328), ACVR2 (NM 001616), ACVR2B (NM 001106), ACVRL1 (NM 000020), ADCK1 (NM 020421), ADCK2 (NM 052853), ADCK4 (NM 024876), ADCK5 (NM 174922), ADRBK1 (NM 001619), ADRBK2 (NM 005160), AKT1 (NM 005163), AKT2 (NM 001626), AKT3 (NM 005465), ALK (NM 004304), ALK7 (NM 145259), ALS2CR2 (NM 018571), ALS2CR7 (NM 139158), AMHR2 (NM 020547), ANKK1 (NM 178510), ANKRD3 (NM 020639), APEG1 (NM 005876), ARAF (NM 001654), ARK5 (NM 014840), ATM (NM 000051), ATR (NM 001184), AURKA (NM 003600), AURKB (NM 004217), AURKC (NM 003160), AXL (NM 001699), BCKDK (NM 005881), BCR (NM 004327), BIKE (NM 017593), BLK (NM 001715), BMPR1A (NM 004329), BMPR1B (NM 001203), BMPR2 (NM 001204), BMX (NM 001721), BRAF (NM 004333), BRD2 (NM 005104), BRD3 (NM 007371), BRD4 (NM 014299), BRDT (NM 001726), BRSK1 (NM 032430), BRSK2 (NM 003957), BTK (NM 000061), BUB1 (NM 004336), BUB1B (NM 001211), CABC1 (NM 020247), CAMK1 (NM 003656), CaMK1b (NM 198452), CAMK1D (NM 020397), CAMK1G (NM 020439), CAMK2A (NM 015981), CAMK2B (NM 001220), CAMK2D (NM 001221), CAMK2G (NM 001222), CAMK4 (NM 001744), CAMKK1 (NM 032294), CAMKK2 (NM 006549), CASK (NM 003688), CCRK (NM 012119), CDC2 (NM 001786), CDC2L1 (NM 001787), CDC2L5 (NM 003718), CDC42BPA (NM 014826), CDC42BPB (NM 006035), CDC7L1 (NM 003503), CDK10 (NM 003674), CDK11 (NM 015076), CDK2 (NM 001798), CDK3 (NM 001258), CDK4 (NM 000075), CDK5 (NM 004935), CDK6 (NM 001259), CDK7 (NM 001799), CDK8 (NM 001260), CDK9 (NM 001261), CDKL1 (NM 004196), CDKL2 (NM 003948), CDKL3 (NM 016508), CDKL4 (NM 001009565), CDKL5 (NM 003159), CHEK1 (NM 001274), CHUK (NM 001278), CIT (NM 007174), CLK1 (NM 004071), CLK2 (NM 003993), CLK3 (NM 003992), CLK4 (NM 020666), CRK7 (NM 016507), CSF1R (NM 005211), CSK (NM 004383), CSNK1A1 (NM 001892), CSNK1D (NM 001893), CSNK1E (NM 001894), CSNK1G1 (NM 022048), CSNK1G2 (NM 001319), CSNK1G3 (NM 004384), CSNK2A1 (NM 001895), CSNK2A2 (NM 001896), DAPK1 (NM 004938), DAPK2 (NM 014326), DAPK3 (NM 001348), DCAMKL1 (NM 004734), DCAMKL2 (NM 152619), DCAMKL3 (XM 047355), DDR1 (NM 013993), DDR2 (NM 006182), DMPK (NM 004409), DMPK2 (NM 017525.1), DYRK1A (NM 001396), DYRK1B (NM 006484), DYRK2 (NM 006482), DYRK3 (NM 003582), DYRK4 (NM 003845), EEF2K (NM 013302), EGFR (NM 005228), EIF2AK3 (NM 004836), EIF2AK4 (NM 001013703), EPHA1 (NM 005232), EPHA10 (NM 001004338), EPHA2 (NM 004431), EPHA3 (NM 005233), EPHA4 (NM 004438), EPHA5 (NM 004439), EPHA6 (XM 114973), EPHA7 (NM 004440), EPHA8 (NM 020526), EPHB1 (NM 004441), EPHB2 (NM 017449), EPHB3 (NM 004443), EPHB4 (NM 004444), EPHB6 (NM 004445), ERBB2 (NM 004448), ERBB3 (NM 001982), ERBB4 (NM 005235), ERK8 (NM 139021), ERN1 (NM 001433), ERN2 (NM 033266), FASTK (NM 025096), FER (NM 005246), FES (NM 002005), FGFR1 (NM 000604), FGFR2 (NM 022970), FGFR3 (NM 000142), FGFR4 (NM 022963), FGR (NM 005248), FLJ23074 (NM 025052), FLJ23119 (NM 024652), FLJ23356 (NM 032237), FLT1 (NM 002019), FLT3 (NM 004119), FLT4 (NM 002020), FRAP1 (NM 004958), FRK (NM 002031), FYN (NM 002037), GAK (NM 005255), GPRK5 (NM 005308), GPRK6 (NM 002082), GPRK7 (NM 139209), GRK4 (NM 005307), GSG2 (NM 031965), GSK3A (NM 019884), GSK3B (NM 002093), GUCY2C (NM 004963), GUCY2D (NM 000180), GUCY2F (NM 001522), H11 (NM 014365), HAK (NM 052947), HCK (NM 002110), HIPK1 (NM 152696), HIPK2 (NM 022740), HIPK3 (NM 005734), HIPK4 (NM 144685), HR1 (NM 014413), HUNK (NM 014586), ICK (NM 016513), IGF1R(NM 000875), IKBKB (NM 001556), IKBKE (NM 014002), ILK (NM 004517), INSR (NM 000208), INSRR (NM 014215), IRAK1 (NM 001569), IRAK2 (NM 001570), IRAK3 (NM 007199), IRAK4 (NM 016123), ITK (NM 005546), JAK1 (NM 002227), JAK2 (NM 004972), JAK3 (NM 000215), KDR (NM 002253), KIS (NM 144624), KIT (NM 000222), KSR (XM 290793), KSR2 (NM 173598), LAK (NM 025144), LATS1 (NM 004690), LATS2 (NM 014572), LCK (NM 005356), LIMK1 (NM 016735), LIMK2 (NM 005569), LMR3 (XM 055866), LMTK2 (NM 014916), LOC149420 (NM 152835), LOC51086 (NM 015978), LRRK2 (XM 058513), LTK (NM 002344), LYN (NM 002350), MAK (NM 005906), MAP2K1 (NM 002755), MAP2K2 (NM 030662), MAP2K3 (NM 002756), MAP2K4 (NM 003010), MAP2K5 (NM 002757), MAP2K6 (NM 002758), MAP2K7 (NM 005043), MAP3K1 (XM 042066), MAP3K10 (NM 002446), MAP3K11 (NM 002419), MAP3K12 (NM 006301), MAP3K13 (NM 004721), MAP3K14 (NM 003954), MAP3K2 (NM 006609), MAP3K3 (NM 002401), MAP3K4 (NM 005922), MAP3K5 (NM 005923), MAP3K6 (NM 004672), MAP3K7 (NM 003188), MAP3K8 (NM 005204), MAP3K9 (NM 033141), MAP4K1 (NM 007181), MAP4K2 (NM 004579), MAP4K3 (NM 003618), MAP4K4 (NM 145686), MAP4K5 (NM 006575), MAPK1 (NM 002745), MAPK10 (NM 002753), MAPK11 (NM 002751), MAPK12 (NM 002969), MAPK13 (NM 002754), MAPK14 (NM 001315), MAPK3 (NM 002746), MAPK4 (NM 002747), MAPK6 (NM 002748), MAPK7 (NM 002749), MAPK8 (NM 002750), MAPK9 (NM 002752), MAPKAPK2 (NM 032960), MAPKAPK3 (NM 004635), MAPKAPK5 (NM 003668), MARK (NM 018650), MARK2 (NM 017490), MARK3 (NM 002376), MARK4 (NM 031417), MAST1 (NM 014975), MAST205 (NM 015112), MAST3 (XM 038150), MAST4 (XM 291141), MASTL (NM 032844), MATK (NM 139355), MELK (NM 014791), MERTK (NM 006343), MET (NM 000245), MGC33182 (NM 145203), MGC42105 (NM 153361), MGC43306 (C9orf96), MGC8407 (NM 024046), MIDORI (NM 020778), MINK (NM 015716), MKNK1 (NM 003684), MKNK2 (NM 017572), MLCK (NM 182493), MLK4 (NM 032435), MLKL (NM 152649), MOS (NM 005372), MST1R (NM 002447), MST4 (NM 016542), MUSK (NM 005592), MYLK (NM 053025), MYLK2 (NM 033118), MYO3A (NM 017433), MYO3B (NM 138995), NEK1 (NM 012224), NEK10 (NM 152534), NEK11 (NM 024800), NEK2 (NM 002497), NEK3 (NM 002498), NEK4 (NM 003157), NEK5 (MGC75495), NEK6 (NM 014397), NEK7 (NM 133494), NEK8 (NM 178170), NEK9 (NM 033116), NLK (NM 016231), NPR1 (NM 000906), NPR2 (NM 003995), NRBP (NM 013392), NRBP2 (NM 178564), NRK (NM 198465), NTRK1 (NM 002529), NTRK2 (NM 006180), NTRK3 (NM 002530), OBSCN (NM 052843), OSR1 (NM 005109), PACE-1 (NM 020423), PAK1 (NM 002576), PAK2 (NM 002577), PAK3 (NM 002578), PAK-4 (NM 005884), PAK6 (NM 020168), PAK7 (NM 020341), PASK (NM 015148), PCTK1 (NM 006201), PCTK2 (NM 002595), PCTK3 (NM 212503), PDGFRA (NM 006206), PDGFRB (NM 002609), PDK1 (NM 002610), PDK2 (NM 002611), PDK3 (NM 005391), PDK4 (NM 002612), PDPK1 (NM 002613), PFTK1 (NM 012395), PHKG1 (NM 006213), PHKG2 (NM 000294), PIK3R4 (NM 014602), PIM1 (NM 002648), PIM2 (NM 006875), PIM3 (NM 001001852), PINK1 (NM 032409), PKE (NM 173575), PKMYT1 (NM 004203), pknbeta (NM 013355), PLK (NM 005030), PLK3 (NM 004073), PRKAA1 (NM 006251), PRKAA2 (NM 006252), PRKACA (NM 002730), PRKACB (NM 002731), PRKACG (NM 002732), PRKCA (NM 002737), PRKCB1 (NM 002738), PRKCD (NM 006254), PRKCE (NM 005400), PRKCG (NM 002739), PRKCH (NM 006255), PRKCI (NM 002740), PRKCL1 (NM 002741), PRKCL2 (NM 006256), PRKCM (NM 002742), PRKCN (NM 005813), PRKCQ (NM 006257), PRKCZ (NM 002744), PRKD2 (NM 016457), PRKDC (NM 006904), PRKG1 (NM 006258), PRKG2 (NM 006259), PRKR (NM 002759), PRKWNK1 (NM 018979), PRKWNK2 (NM 006648), PRKWNK3 (NM 020922), PRKWNK4 (NM 032387), PRKX (NM 005044), PRKY (NM 002760), PRPF4B (NM 003913), PSKH1 (NM 006742), PSKH2 (NM 033126), PTK2 (NM 005607), PTK2B (NM 004103), PTK6 (NM 005975), PTK7 (NM 002821), PTK9 (NM 002822), PTK9L (NM 007284), PXK (NM 017771), QSK (NM 025164), RAD53 (NM 007194), RAF1 (NM 002880), RAGE (NM 014226), RET (NM 020975), RHOK (NM 002929), RIOK1 (NM 031480), RIOK2 (NM 018343), RIPK1 (NM 003804), RIPK2 (NM 003821), RIPK3 (NM 006871), RIPK5 (NM 015375), RNASEL (NM 021133), ROCK1 (NM 005406), ROCK2 (NM 004850), ROR1 (NM 005012), ROR2 (NM 004560), ROS1 (NM 002944), RPS6KA1 (NM 002953), RPS6KA2 (NM 021135), RPS6KA3 (NM 004586), RPS6KA4 (NM 003942), RPS6KA5 (NM 004755), RPS6KA6 (NM 014496), RPS6 KB1 (NM 003161), RPS6 KB2 (NM 003952), RPS6KC1 (NM 012424), RPS6KL1 (NM 031464), RYK (NM 002958), SBK (XM 370948), SCYL1 (NM 020680), SCYL2 (NM 017988), SGK (NM 005627), SgK069 (SU SgK069), SgK085 (XM 373109), SgK110 (SU SgK110), SGK2 (NM 016276), SgK223 (XM 291277), SgK269 (XM 370878), SgK424 (CGP SgK424), SgK493 (SU_SgK493), SgK494 (NM 144610), SgK495 (NM 032017), SGKL (NM 013257), SK681 (NM 001001671), SLK (NM 014720), SMG1 (NM 015092), SNARK (NM 030952), SNF1LK (NM 173354), SNF1LK2 (NM 015191), SNK (NM 006622), SNRK (NM 017719), SRC (NM 005417), SRMS (NM 080823), SRPK1 (NM 003137), SRPK2 (NM 003138), SSTK (NM 032037), STK10 (NM 005990), STK11 (NM 000455), STK16 (NM 003691), STK17A (NM 004760), STK17B (NM 004226), STK18 (NM 014264), STK19 (NM 032454), STK22B (NM 053006), STK22C (NM 052841), STK22D (NM 032028), STK23 (NM 014370), STK24 (NM 003576), STK25 (NM 006374), STK3 (NM 006281), STK31 (NM 031414), STK32B (NM 018401), STK33 (NM 030906), STK35 (NM 080836), STK36 (NM 015690), STK38 (NM 007271), STK38L (NM 015000), STK39 (NM 013233), STK4 (NM 006282), STLK5 (NM 001003787), STYK1 (NM 018423), SUDD (NM 003831), SYK (NM 003177), TAF1 (NM 138923), TAF1L (NM 153809), TAO1 (NM 004783), TAOK1 (NM 020791), TAOK3 (NM 016281), TBCK (NM 033115), TBK1 (NM 013254), TEC (NM 003215), TEK (NM 000459), TESK1 (NM 006285), TESK2 (NM 007170), TEX14 (NM 031272), TGFBR1 (NM 004612), TGFBR2 (NM 003242), TIE (NM 005424), TIF1 (NM 003852), TLK1 (NM 012290), TLK2 (NM 006852), TNIK (NM 015028), TNK1 (NM 003985), TOPK (NM 018492), TP53RK (NM 033550), TRAD (NM 007064), TRIB1 (NM 025195), TRIB2 (NM 021643), TRIB3 (NM 021158), TRIM28 (NM 005762), TRIM33 (NM 015906), TRIO (NM 007118), TRPM6 (NM 017662), TRPM7 (NM 017672), TRRAP (NM 003496), TSSK4 (NM 174944), TTBK1 (NM 032538), TTBK2 (NM 173500), TTK (NM 003318), TTN (NM 003319), TXK (NM 003328), TYK2 (NM 003331), TYRO3 (NM 006293), ULK1 (NM 003565), ULK2 (NM 014683), ULK3 (NM 015518), ULK4 (NM 017886), VRK1 (NM 003384), VRK2 (NM 006296), VRK3 (NM 016440), WEE1 (NM 003390), Wee1B (NM 173677), YANK1 (NM 145001), YES1 (NM 005433), ZAK (NM 016653), and/or ZAP70 (NM 001079).

The term “allergen” in the course of the present invention refers to complete or partial antigens as defined herein that elicit hypersensitivity and/or allergic reactions. Examples are Der p 5 (mite), Bet v 1 (birch pollen), Phl p 1 (grass pollen), Asp f l/a (Aspergillus), PLA 2 (bee), Hev b (latex). (Schmid-Grendelmeier and Crameri, Recombinant allergens for skin testing. Int Arch Allergy Immunol 2001, 125, 96-111)

Antigens of microbial and eukaryotic pathogens and of cancer testis antigens are enclosed in the list above.

In yet another preferred embodiment, the at least one protein toxin and/or at least one protein toxin subunit is selected from the group consisting of: bacterial toxin, enterotoxin, exotoxin, type I toxin, type II toxin, type III toxin, type IV toxin, type V toxin, RTX toxin, AB toxin, A-B toxin, A/B toxin, A+B toxin, A-5B toxin and/or AB5 toxin.

In yet another preferred embodiment, at least one protein toxin and/or at least one protein toxin subunit is selected from the group consisting of: Adenylate cyclase toxin, Anthrax toxin, Anthrax toxin (EF), Anthrax toxin (LF), Botulinum toxin, Cholera toxin (CT, Ctx), Cholera toxin subunit B (CTB, CtxB), Diphtheria toxin (DT, Dtx), E. coli LT toxin, E. coli heat labile enterotoxin (LT), E. coli heat labile enterotoxin subunit B (LTB), E. coli ST toxin, E. coli heat stabile enterotoxin (ST), Erythrogenic toxin, Exfoliatin toxin, Exotoxin A, Perfringens enterotoxin, Pertussis toxin (PT, Ptx), Shiga toxin (ST, Stx), Shiga toxin subunit B (STB, StxB), Shiga-like toxin, Staphylococcus enterotoxins, Tetanus toxin (TT), Toxic shock syndrome toxin (TSST-1), Vero toxin (VT), Toxin A (TA) and Toxin B (TB) of Clostridium difficile, Lethal Toxin (LT) and Hemorrhagic Toxin (HT) of Clostridium sordellii, alpha Toxin (AT) of Clostridium novyi.

In yet a further preferred embodiment, the at least one complete or partial antigen of at least one wild-type or mutated protein and the at least one protein toxin and/or at least one protein toxin subunit are linked together to enable the expression and/or secretion of a fusion protein encoded by both components.

In yet a further preferred embodiment, the fusion protein is selected from the group consisting of: CtxB-PSA, CtxB-B-Raf V600E KD, CtxB-B-Raf V600E kinase domain, CtxB-B-Raf V600E kinase domain KD, CtxB-B-Raf, CtxB-B-Raf KD, CtxB B-Raf kinase domain KD, CtxB-HA1, CtxB-HA12C.

Secretion is the process of segregating, elaborating, and releasing chemicals from a cell, or a secreted chemical substance or amount of substance. Secretion is not unique to eukaryotes alone; it is present in bacteria and archaea as well. ATP binding cassette (ABC) type transporters are common to all the three domains of life. The Sec system is also another conserved secretion system which is homologous to the translocon in the eukaryotic endoplasmic reticulum consisting of Sec 61 translocon complex in yeast and Sec Y-E-G complex in bacteria. Gram-negative bacteria have two membranes, thus making secretion topologically more complex. So there are at least five specialized secretion system in Gram negative bacteria:

(1) Type I secretion system: It is same as the ATP binding cassette transporters mentioned above.

(2) Type II secretion system: It depends on the Sec system for a protein to cross the inner membrane and another special system to cross the outer membrane. Bacterial pili use modifications of the sec system, but are different from type I system.

(3) Type III secretion system (T3SS): It is homologous to bacterial flagellar basal body. It is like a molecular syringe through which a bacterium (e.g. Shigella or Yersinia) can inject proteins into eukaryotic cells. The low Ca²⁺ concentration in the cytosol opens the gate that regulates T3SS. The Hrp system in plant pathogens injects hairpins through similar mechanisms into plants.

(4) Type IV secretion system: It is homologous to conjugation machinery of bacteria (and archaeal flagella). It is capable of transporting both DNA and proteins. It was discovered in Agrobacterium tumefaciens, which uses this system to introduce the Ti plasmid and proteins into the host which develops the crown gall (tumor). Helicobactor pylori uses a type IV secretion system to inject Cag A into gastic epithelial cells. Bordetella pertussis, the causative agent of whooping cough, secretes the pertussis toxin partly through the type IV system.

(5) Type V secretion system, also called autotransporter system: This use the sec system for crossing the inner membrane. The proteins which use this path have the capability to form a beta barrel in their C terminus and insert into the outer membrane to transport the rest of the peptide out. Finally the beta barrel may be cleaved and left back in the outer membrane. Some people believe these remnants of the autotransporters gave rise to the porins which are similar beta barrels.

Bacteria as well as mitochondria and chloroplasts also use many other special transport systems such as the twin-arginine translocation (Tat) pathway which, in contrast to Sec-dependent export, transports fully folded proteins across the membrane. The name of the system comes from the requirement for two consecutive arginines in the signal sequence required for targeting to this system. Secretion in gram-negative bacteria involves overcoming the inner and outer membrane by the way of a suitable secretion system, like e.g. the Hly type I or type III secretion system or AIDA auto-transporter. In gram-positive bacteria the secretion system has to overcome the inner membrane and the cell wall, which, in most strains, can be achieved by fusion with a suitable secretion signal.

In another aspect, the object of the present invention has been surprisingly solved by providing a process for the production of a recombinant bacterium according to above aspects and embodiments, comprising the steps
(a) transforming a bacterium with at least one nucleotide sequence coding for the E. coli hemolysin secretion system, wherein the at least one nucleotide sequence comprises full length or partial HlyA, HlyB and HlyD gene sequences under control of the hly-specific promoter or a not hly-specific bacterial promoter, wherein the at least one nucleotide sequence is integrated into the bacterial chromosome or, preferably, is located on a plasmid,
(b) complementing the bacterium of step a) with at least one nucleotide sequence coding for a protein that effects an increased expression and/or increased secretion of full length or partial HlyA compared to normal/wild-type HlyA expression and/or secretion, where the at least one nucleotide sequence preferably comprises rfaH and/or rpoN gene and is integrated into the bacterial chromosome or, preferably, is located on a plasmid
(c) optionally, deleting or inactivating rpoS gene in a bacterium of step b)
(d) optionally, attenuating the bacterium of step b) or c), preferably by deleting or inactivating at least one gene selected from the group consisting of: aroA, aro, asd, gal, pur, cya, crp, phoP/Q, omp.
(e) optionally, transforming the bacterium of step b), c), or d) with at least one nucleotide sequence coding for at least one complete or partial antigen of at least one wild-type or mutated protein and at least one nucleotide sequence coding for at least one protein toxin and/or at least one protein toxin subunit, where the at least one nucleotide sequence is integrated into the bacterial chromosome or, preferably, is located on a plasmid.

In another aspect, the object of the present invention has been surprisingly solved by providing a pharmaceutical composition comprising at least one recombinant bacterium, preferably at least one lyophilized recombinant bacterium, according to above aspects and embodiments and a pharmaceutically acceptable carrier, preferably capsules.

In another aspect, the object of the present invention has been surprisingly solved by providing a medicament comprising at least one recombinant bacterium according to above aspects and embodiments or a pharmaceutical composition according to above aspects and embodiments.

In another aspect, the object of the present invention has been surprisingly solved by providing a medicament comprising at least one recombinant bacterium according to above aspects and embodiments or a pharmaceutical composition according to above aspects and embodiments for the treatment and/or prophylaxis of physiological and/or pathophysiological conditions selected from the group consisting of: diseases involving macrophage inflammations where macrophages are associated with disease onset or disease progression, tumor diseases, uncontrolled cell division, malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, oesophageal cancer, thyroid cancer, osteosarcomas, retinoblastomas, thymoma, testicular cancer, lung cancer, bronchial carcinomas, breast cancer, mamma carcinomas, intestinal cancer, colorectal tumors, colon carcinomas, rectum carcinomas, gynecological tumors, ovary tumors/ovarian tumors, uterine cancer, cervical cancer, cervix carcinomas, cancer of body of uterus, corpus carcinomas, endometrial carcinomas, urinary bladder cancer, bladder cancer, skin cancer, basaliomas, spinaliomas, melanomas, intraocular melanomas, leukemia, chronic leukemia, acute leukemia, lymphomas, infection, viral or bacterial infection, influenza, chronic inflammation, organ rejection, autoimmune diseases, diabetes and/or diabetes type II.

In another aspect, the object of the present invention has been surprisingly solved by providing a pharmaceutical kit comprising at least one recombinant bacterium according to above aspects and embodiments or a pharmaceutical composition according to above aspects and embodiments or a medicament according to above aspects and embodiments and a pharmacologically acceptable buffer, preferably a carbonate buffer.

In the course of the invention the term “auxotrophic bacterium” refers to a bacterium carrying at least one mutation which leads to a reduced growth rate in the infected host.

In the course of the invention the term “attenuated bacterium” refers to a bacterium, which is attenuated in its virulence either by a loss of function in at least one virulence factor necessary for infection of the host and/or by an auxotrophic mutation leading to an impaired growth within the host, i.e. the virulence is reduced compared to the non-attenuated wild-type counterpart, for instance a bacterium that carries a deleted or inactivated aroA, aro, asd, gal, pur, cya, crp, phoP/Q, omp gene or is a temperature-sensitive mutant or an antibiotic-dependent mutant (Cardenas L. and Clements J. D. Clin Microbiol Rev 1992; 5: 328-342).

The term “recombinant DNA” in the course of the present invention refers to artificial DNA which is molecular-genetically engineered through the combination or insertion or deletion of one or more (parts of) DNA strands, thereby combining DNA sequences which would not normally occur together in nature. In terms of genetic modification, recombinant DNA is produced through the addition of relevant DNA into an existing organismal genome or deletion of relevant DNA in an existing organismal genome, such as the chromosome and/or plasmids of bacteria, to code for or alter different traits for a specific purpose, such as immunity. It differs from genetic recombination, in that it does not occur through processes within the cell or ribosome, but is exclusively molecular-genetically engineered.

The term “recombinant plasmid” in the course of the present invention refers to recombinant DNA which is present in the form of a plasmid.

The term “recombinant bacterium” in the course of the present invention refers to a bacterium harboring recombinant DNA and/or recombinant plasmid(s) and/or non-recombinant DNA artificially introduced into such bacterium.

The term “nucleotide sequence” in the course of the present invention refers to dsDNA, ssDNA, dsRNA, ssRNA or dsDNA/RNA hybrids. Preferred is dsDNA.

The term “epigenetic changes” in the course of the present invention refers to changes on the DNA level, i.e. by DNA methylation or demethylation, binding poly-comb proteins, histone acylation etc. which influence the expression level of at least one gene.

The term “regulatory DNA” in the course of the present invention refers to regions in the DNA which influence the expression of at least one gene by binding of regulatory proteins or by inducing epigenetic changes.

The term “spp.” in connection with any bacterium is intended to comprise for the purpose of the present invention all members of a given genus, including species, subspecies and others. The term “Salmonella spp.” for instance is intended to comprise all members of the genus Salmonella, such as Salmonella typhi and Salmonella typhimurium.

The term “antigen” in the course of the present invention refers to molecules that react with antibodies, i.e. that are able to generate antibodies. Some antigens do not, by themselves, elicit antibody production; only those that can induce antibody production are called immunogens. For the purpose of the present invention, all kinds of known antigens are intended to be comprised. It is within the knowledge of a person skilled in the art to retrieve the necessary information about potential antigens by means of databases and/or experimental screening without undue burden. Examples of antigens are among others cell antigens, tissue-cell specific antigens (e.g. tissue cells from which the tumor derives), cell protein antigens, viral antigens, viral protein antigens and the like. Preferred are protein antigens. Further preferred are heterologous antigens or foreign antigens, i.e. antigens which are not endogenous to the respective microorganism of the invention or antigens which are not expressed by the respective microorganism of the invention by nature, but are introduced into it by means of standard molecular biotechnological methods.

The term “complete antigen” in the course of the present invention refers to complete molecules that react with antibodies according to the definition above. Examples of complete antigens are for instance full-length proteins, which are also preferred.

The term “partial antigen” in the course of the present invention refers to specific parts of molecules that react with antibodies according to the definition above. Partial antigens can be for instance protein motives such as amino acid loops within proteins, protein kinase domains, epitopes and the like. Preferred are protein kinase domains and epitopes, the latter of which are specific sites of an antigen recognized by an antibody (also referred to as antigenic determinants).

The terms “wild-type” and “mutated” in connection with “protein” in the course of the present invention refer to proteins consisting of either their “natural” dominating amino acid sequence (encoded by the respective nucleotide sequence) and proteins that have one or more mutations in their amino acid sequence (encoded by the respective nucleotide sequence) compared to the wild-type sequence, respectively. Preferably wild-type and/or mutated proteins are derived from tumor cells. As for partial antigens it is further preferred that the sequence encompasses mutations, i.e. an epitope is chosen that preferably contains one or more mutations, for instance the B-Raf V600E epitope.

Bacterial infections comprise, but are not limited to, anthrax, bacterial meningitis, botulism, brucellosis, campylobacteriosis, cat scratch disease, cholera, diphtheria, epidemic typhus, impetigo, legionellosis, leprosy (Hansen's disease), leptospirosis, listeriosis, lyme disease, melioidosis, MRSA infection, nocardiosis, pertussis (whooping cough), plague, pneumococcal pneumonia, psittacosis, Q fever, Rocky Mountain Spotted Fever (RMSF), salmonellosis, scarlet fever, shigellosis, syphilis, tetanus, trachoma, tuberculosis, tularemia, typhoid fever, typhus, urinary tract infections, bacterially caused heart diseases.

Viral infections comprise, but are not limited to, HIV, AIDS, AIDS related complex (ARC), chickenpox (varicella), common cold, cytomegalovirus infection, Colorado tick fever, Dengue fever, Ebola haemorrhagic fever, hand, foot and mouth disease, hepatitis, Herpes simplex, Herpes zoster, HPV, influenza (flu), Lassa fever, measles, Marburg haemorrhagic fever, infectious mononucleosis, mumps, poliomyelitis, progressive multifocal leukencephalopathy, rabies, rubella, SARS, smallpox (variola), viral encephalitis, viral gastroenteritis, viral meningitis, viral pneumonia, West Nile disease, Yellow fever.

Chronic inflammations or chronic inflammatory diseases comprise, but are not limited to, chronic cholecystitis, bronchiectasis, rheumatoid arthritis, Hashimoto's thyroiditis, inflammatory bowel disease (ulcerative colitis and Crohn's disease), silicosis and other pneumoconiosis.

Autoimmune diseases comprise, but are not limited to, systemic syndromes, such as SLE, Sjögren's syndrome, scleroderma, rheumatoid arthritis and polymyositis as well as local syndromes, such as IDDM, Hashimoto's thyroiditis, Addison's disease, pemphigus vulgaris, psoriasis, atopic dermatitis, atopic syndrome, asthma, autoimmune haemolytic anaemia, multiple sclerosis.

The above illustrated bacteria as well as the preferred embodiments are herein referred to as bacterium of the invention.

The bacterium of the invention allows higher expression and secretion of hemolysin via a type I secretion system. Furthermore, rfaH mediates a better uptake and faster degradation by macrophages. This leads to a higher immunogenicity against hemolysin, but not lipopolysaccharide (LPS) in the mouse mode. Therefore, the bacterium of the invention is ideally suited for delivering heterologous antigens as described herein to the vaccinee's immune system. For Salmonella typhi Ty21a in particular, type I secretion seems to be the only suitable way for secreting heterologous antigens as this strain lacks functional type III secretion systems. Moreover, type I secretion is less effective in Ty21a than in other Salmonella strains which is circumvented by complementation with factors of rpoS signalling pathway, namely rpoS, rfaH and rpoN.

The attenuated bacteria of the present invention can be administered in a known manner. The route of administration may thereby be any route which effectively transports the bacteria to the appropriate or desired site of action, for example non-orally or orally, in particular intravenously, topically, transdermally, pulmonary, rectally, intravaginally, nasally or parenteral or by implantation. Oral administration is preferred.

Non-oral administration can take place for example by intravenous, subcutaneous, intramuscular injection of sterile aqueous or oily solutions, suspensions or emulsions, by means of implants or by ointments, creams or suppositories. Administration as sustained release form is also possible where appropriate. Implants may comprise inert materials, e.g. biodegradable polymers or synthetic silicones such as, for example, silicone rubber. Intravaginal administration is possible for example by means of vaginal rings. Intrauterine administration is possible for example by means of diaphragms or other suitable intrauterine devices. Transdermal administration is additionally provided, in particular by means of a formulation suitable for this purpose and/or suitable means such as, for example, patches.

Oral administration can take place for example in solid form as tablet, capsule, gel capsule, coated tablet, granulation or powder, but also in the form of a drinkable solution. The compounds of the invention can for oral administration be combined with known and ordinarily used, physiologically tolerated excipients and carriers such as, for example, gum arabic, talc, starch, sugars such as, for example, mannitol, methylcellulose, lactose, gelatin, surface-active agents, magnesium stearate, cyclodextrins, aqueous or nonaqueous carriers, diluents, dispersants, emulsifiers, lubricants, preservatives and flavorings (e.g. essential oils). The bacteria of the invention can also be dispersed in a microparticulate, e.g. nanoparticulate, composition.

A preferred mode of application is oral application. In a preferred embodiment, a Salmonella strain according to this invention is fermented in appropriate medium, harvested and washed by centrifugation and subsequently formulated and stabilized using appropriate substances and lyophylized. The lyophylized substance is filled into stomach resistant capsules containing life cell numbers preferably between 10⁹ to 10¹⁰ bacteria. The capsules are orally uptaken with liquid.

Alternatively, lyophylized bacteria as described above are distributed together with sachets containing buffer which is able to neutralize the stomach acid (pharmaceutical kit). In a preferred embodiment, this buffer is a carbonate buffer. Immediately prior to use, the buffer is prepared with water and taken up, immediately afterwards the lyophylized bacteria are uptaken mixed with water.

Yet another alternative is the use of frozen bacteria. In this case, after washing bacteria are stabilized via a stabilizer, preferably succhrose or glycerine, and subsequently frozen and stored at −80° C. preferably in concentrations between 10 to 10¹¹, preferably 10⁹ to 10¹⁰ bacteria per dose. This preparation is preferably used in a pharmaceutical kit as described above in conjunction with carbonate buffer.

Possible modes of manufacturing the bacteria of the invention are:

Factors enhancing the ability of type I secretion, like rfaH and rpoN, can be introduced into the bacterium of the invention via plasmids or genomic integration. These factors should be overexpressed to achieve an optimal upregulation of the hemolysin determinant or the heterologous antigen-Hly fusion, respectively. Overexpression can be achieved by introducing multiple copies of the gene of interest via plasmids. These plasmids should allow stable expression of their products and stable replication in vitro and in vivo, such as plasmid pBR322. Plasmids can also be stabilized by using balanced-lethal systems, where essential genes are deleted on the chromosome and complemented on the plasmid. Plasmid loss would lead to non-viable bacteria giving a selective pressure for plasmid maintenance. Another way of overexpression is facilitated by integrating stronger or constitutively active promoters, like P_{tac} or Pₜᵣₚ upstream of target genes on the bacterial chromosome. This could be manufactured by homologous recombination.

The contents of all cited references and patents are hereby incorporated by reference. The invention is explained in more detail by means of the following examples without, however, being restricted thereto.

### EXAMPLES

### Material and Methods

### Bacterial Strains and Plasmids:

The bacterial strains and DNAs used are listed in Table 1. Strains were cultivated at 37° C. in Luria Bertani (LB) medium (Sigma, Schnelidorf, Germany) or Brain Heart Infusion (BHI) medium (BD Difco, Sparks, USA). Media were solidified with 1.0% (wt/vol) agar. When required, media were supplemented with ampicillin (Ap; 100 μg/ml) or/and chloramphenicol (Cm; 20 μg/ml). For survival and stress assays, strains were grown routinely at 37° C. in CY medium at pH 7.2 with aeration by vigorous shaking (Yeast extract, 12 mg ml⁻¹, Hy-Case, 20 mg ml⁻¹, pepticase, 12 mg ml⁻¹, NaH₂PO₄, 1.25 mg ml⁻¹; NaCl, 3.3. mg ml⁻¹; glucose, 2 mg ml⁻¹).

Construction of Serotype S. typhi Ty21a RpoS⁺ (rpoSTy21a) and S. typhi Ty21a RfaH⁺ (rfaHTy21a):

The PCR fragment of rpoS was generated with pfU Polymerase (Stratagene, La-Jolla, USA) by the primers rpoS_up (5′CATCGCCTGGATCCCCGGGAACG 3′-SEQ ID NO:1) and rpoS_down (5′ GACGCAAAAAGCTTTTGATGACGCGCC 3′-SEQ ID NO:2) using standard PCR techniques. Chromosomal DNA from S. typhimurium aroA served as template, the annealing temperature was 62° C. the elongation step was taken out for 4 min. The 1.9 kB fragment contains putative promoter regions of rpoS as determined by the Neural Network Promoter Prediction algorithm (http://www.fruitfly.org/seq_tools/promoter.html). RfaH was amplified with Phusion Taq (Finnzymes) with primers rfaH_up (5′ GAGGATCCACAGGAAGCTTGATGCGTTTTAG 3′-SEQ ID NO:3) and rfaH_down (5′CGCAAGATTTAGGGATCCTTCAGAATACGACC 3′-SEQ ID NO:4) using chromosomal DNA from Ty21a as template. The PCR was carried out as follows: 98° C. for 30 s followed by 33 cycles with 98° C./10 s, 60° C./90 s and 72° C. for 20 s.

The amplified genes were digested with BamHI and HindIII and ligated in the vector pACYC184 cut with the same enzymes, giving plasmids pRpoS and pRfaH, respectively. To construct serotype S. typhi Ty21a RpoS⁺ and S. typhi Ty21a RfaH⁺, the plasmid pRpoS and pRfaH were introduced into S. typhi Ty21a by electroporation using a Bio-Rad Gene Pulser (Hercules, Calif., USA) at 2.5 kV, 25 microfarads (μF), and 200 Ohm in a 0.1-cm electroporation cuvette.

Expression of rpoS was assayed in the rpoS-negative S. typhi Ty21a strain by a positive catalase reaction. RpoS positive clones can be detected by producing visible air bubbles when treated with Hydrogen peroxide. The extent of bubbling indicated absence or reduction of catalase production in strains with differing rpoS genotypes [14].

### Oxidative Stress Test:

For the oxidative stress test, bacterial strains were incubated in CY-medium to stationary phase. The cells were harvested by centrifugation, washed with 0.9% NaCl and equally separated into two tubes. Bacteria were then suspended in CY medium with none, 3 mM or 30 mM H₂O₂. Survival was assessed after 20 min incubation at 37° C. by plating 0.1 ml cell suspension on BHI-agar and overnight incubation at 37° C. The percentage of surviving bacteria was determined by comparing the numbers of colony forming units (CFU) of H₂O₂ treated versus untreated cells.

### Semi-Quantitative Real-Time RT-PCR:

Whole RNA was extracted from 5×10⁹ and 1×10¹⁰ bacteria at OD₆₀₀=0.4 and OD₆₀₀=2.5 respectively with RNAeasy mini kit (Qiagen, Hilden, Germany). On column DNA digestion with RNase-Free DNase kit (Qiagen, Hilden, Germany) was performed for 20 min at room temperature. Presence of DNA was analysed by PCR with the primers htrB_up (5′GCGAGAATACGGAGAATTG 3′-SEQ ID NO:5) and htrB2_down (5′ GAGGGGAAAAATTGCAG 3′-SEQ ID NO:6). Residual DNA was digested with DNAfree kit (Ambion, Austin, Tex.). 0.5 μg of total RNA were applied for cDNA synthesis with random hexamers using First Strand cDNA Synthesis Kit (Fermentas, Burlington, Canada). Primers were used as follows.

The primers Cat RT (F) and Cat RT (R) were used to amplify a 136 bp fragment specific for the cat gene. The primers HlyA RT (F) and HlyA RT (R) were used to amplify a 121 bp fragment specific for the hlyA gene. The primers HlyD RT (F) and HlyD RT (R) were used to amplify a 135 bp fragment specific for the hlyD gene. The primers RfaH RT (F) and RfaH RT (R) were used to amplify a 91 bp fragment specific for the rfaH gene

A quantitative real-time PCR (qRT-PCR) was performed on the Rotor-Gene (Corbett, Sydney, Australia) using DyNAmo™ HS SYBR® Green qPCR Kit (Finnzymes, Espo, Finnland). In a total volume of 20 μl each sample was analysed in triplicate, each qRT-PCR was performed in duplicate. 1 μl of tenfold diluted cDNA was used for qRT-PCR. qRT-PCR conditions were as follows: step 1-900 s 95° C.; step 2—cycle 40×: 94° C. for 10 s, 57° C. for 20 s, 72° C. for 30 s; step 3-72° C. for 300 s; step 4-25° C. for 600 s; step 5—melt curve between 70° C. and 95° C. The presence of the primer specific amplicon was determined by detection of one melting-temperature peak and a single band at the expected size on a 2% agarose gel after electrophoresis.

The Ct values and determined with the Rotor-Gene Analysis Software V4.6.70 (Corbett, Sydney, Australia). By raising 2 by the power of the corresponding Ct value a relative unit for comparison of the initial RNA amount was calculated. The relative changes in gene expression between different strains were calculated after normalization with the cat gene as internal control. Significances of regulation were calculated using Students T-Test.

### SDS PAGE and Western Blot:

Bacteria were grown in BHI medium containing appropriate antibiotics. At different time points, 20 ml of the culture were taken and centrifuged for 30′ at 4000 rpm and 4° C. in a Hereaus centrifuge. The Pellet was lysed in 5× Laemmli-buffer and referred to as cellular proteins. 20 ml of the supernatant were transferred into a fresh tube. Subsequently, 2 ml TCA (trichlor-acetic acid, Applichem, Darmstadt, Germany) were added; the liquid was mixed and incubated on ice overnight. After incubation, the suspension was centrifuged for 1 h at 4000 rpm and 4° C. in a Hereaus centrifuge. The supernatant was decanted and the pellet was washed with 1 ml Aceton p.a. (Applichem, Darmstadt, Germany); the precipitate was centrifuged for 10′ at 4000 rpm and 4° C. in a Hereaus centrifuge. The pellet was air-dried and taken up in 150 μl 5× Laemmli-buffer with or without R-mercaptoethanol [15] and pH neutralized by adding 1 μl of saturated Tris solution. 20-25 μl of the supernatant fraction were used for each lane in SDS PAGE. The separated proteins were electrophoretically transferred to Hybond ECL nitrocellulose membrane (Amersham-Pharmacia, Little Chalfont, U.K.) and blocked for 1 h with PBS containing 5% skimmed milk. The membrane was washed in PBS-Tween 0.05%, incubated with HlyAs antibody [12, 16] and subsequently incubated with HRP-coupled anti rabbit IgG ( 1/1,000; Dianova, Hamburg, Germany) for 1 h. The Western blot was carried out using the enhanced chemiluminescence kit (GE Healthcare Life Science, Munich, Germany).

Purification of LPS from Ty21a:

After harvest by centrifugation the Ty21a cells were disintegrated mechanically in the Dyno-Mill (Bachofen, Basel, Switzerland) using glass beads and PBS. Cell debris and cytoplasm were separated from the glass beads using a G-1 sintered glass filter. The glass beads were washed twice with PBS. Filtrate and wash buffer were pooled and then centrifuged (20000×g, 4° C., 60 min). The sediment containing the cell wall debris was suspended in water and homogenized.

The LPS were then isolated by the hot phenol extraction [17] from the cell wall fraction which was mixed in a ratio of 1:1.28 with a 75° C. hot 80% phenol solution. After cooling to RT the water phase was separated from the phenol. The latter was extracted again with water as described above. After the phenol treatment the combined water phases were dialysed against water for removing residual phenol.

The LPS of the water fraction were sedimented by ultracentrifugation at 150000×g for a minimum of 3 hours at 4° C. The sediment was suspended in water and homogenized and further purified by at least two more ultracentrifugations. The sediment was suspended and homogenized in water and then controlled by UV spectroscopy on absence of proteins and nucleic acids [18].

### Salmonella Infection of Macrophages, Invasion and Survival Assay:

For infection of RAW 264.7 cells, bacteria were grown to stationary phase and washed in PBS. The bacteria were diluted in RPMI 1640 medium and then added to the cells seeded in 24-well tissue culture plates at a multiplicity of infection of 100. The bacteria were centrifuged onto the cells at 500×g for 5 min and then incubated for 2 h at 37° C. in an atmosphere of 5% CO₂. After infection, the macrophages were washed two times with PBS and then incubated in RPMI 1640 containing 100 μg of gentamicin/ml (Sigma, Schnelldorf, Germany). After 2 h and 4 h of incubation, the macrophages were washed with PBS and lysed with 1% Triton X-100. For enumeration of intracellular bacteria, serial dilutions were plated on LB agar plates.

Intranasal Immunization (i.n.) of Balb/c Mice with Different S. typhi Ty21a Strains:

Infection aliquots were prepared by cultivating the strains overnight at 37° C. in BHI medium containing appropriate antibiotics. The next day, cells were harvested by centrifugation in a Beckmann-Coulter centrifuge, washed in PBS and concentrated 200 fold in PBS containing 20% Glycerol and aliquoted in 500 μl portions. Aliquots were stored at least 24 hours at −80° C. before the CFU was determined by plating serial dilutions on BHI agar plates. The vials were thawed on ice 30 min prior to use. Six to eight weeks old C57BL/6 mice (Harlan-Winkelmann, Borchem, Germany) were immunized i.n. twice with 8×10⁸ salmonellae in 10-15 μl from the infection aliquots on days 0 and 28. The vaccine was applied using a micropipette into the nares of mice without anesthesia. The mice were sacrificed 21 days after the second immunization and the sera were analyzed for specific antibodies against LPS and HlyA.

### Analysis of Antibody Response Against Hemolysin and LPS by ELISA:

The titers of hemolysin or LPS antibodies present in mouse sera were determined by ELISA. For detection of LPS antibodies, 1 μg/ml S. typhi lipopolysaccharide (LPS, Berna Biotech Ltd, Berne, Switzerland) were coated onto NUNC 96-Well MaxiSorp plates (Nunc A/S, Kamstrup, Denmark) at 4° C. overnight. For detection of HlyA specific antibodies, HlyA was precipitated from culture supernatants using strain S. typhi Ty21a+pANN202-806 following the protocol in the SDS PAGE section. Instead of Laemmli-buffer, the pellet was resuspended in PBS and neutralized with saturated Tris solution. Finally, the solution was diluted 1:500 in coating buffer (Carbonate Buffer, pH 9.6) and coated overnight on the same 96-well plates.

Plates were washed twice with washing buffer (0.05% Tween (Sigma) in PBS) and blocked with 1% BSA (Sigma) in PBS. After washing twice, three dilutions of mouse sera (1:33; 1:100; 1:300) in 100 μl conjugate buffer (1% BSA, 0.05% Tween in PBS) were incubated in duplicates for 1.5 h at 37° C. After four washing steps, alkaline phosphatase coupled sheep anti-mouse IgG or anti mouse IgG and IgM (Dianova, Hamburg, Germany) diluted 1:1000 in 100 μl conjugate buffer was added. After 1 h at 37° C. and two washing steps, 50 μl of pNPP (Sigma) substrate in buffer was added. The reaction was incubated at room temperature and stopped after 30 min by 50 μl 1M NaOH. Optical density (OD) was read at a wavelength of 405 nm in a TECAN Spectra Thermo microplate reader (TECAN, Grödig, Austria).

### Results:

### 1. Hemolysin Secretion in Different Vaccine Salmonella Strains

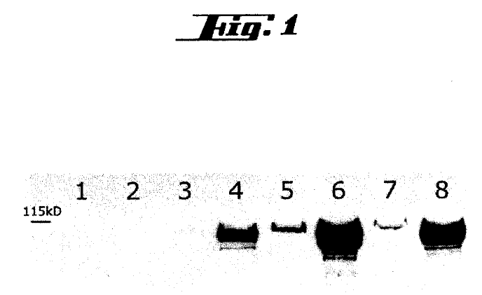

By the analysis of the hemolysin secretion efficiency in different attenuated vaccine Salmonella strains it was found that the amount of secreted protein in Ty21a is less than that of all other tested strains under the same conditions (FIG. 1). The vaccine strain Ty21a is an attenuated mutant strain of S. typhi Ty2, achieved by multiple mutations induced by chemical mutagenesis [1]. Therefore some of the mutations could be responsible for this effect. In order to test this supposition an approach for testing the effect of single mutations in Ty21a on hemolysin secretion was started.

2. Analysis of the Effect of galE on Hemolysin Expression and Secretion in Ty21a

The decisive characteristic of strain Ty21a is a complete deficiency of the enzyme uridine diphosphate (UDP)-galactose-4-epimerase activity. The strain resulted from experiences with selection of galE mutants that were found to have reduced activities of the other enzymes of the galactose pathway, namely galactose permease, galactokinase, and galactose-1-phosphat-uridyl transferase [2]. Virulence as well as the capability to excite an adequate immune response of galE mutants depend on the activity of all enzymes responsible for metabolism of galactose and its distribution within the bacterial cell [2]. As a result of the galE mutation, Ty21a is a rough-type strain due to the formation of lipopolysaccharide (LPS) without part of the corepolysaccharide and the O-antigen, which turned out to be the main antigenic determinant on the cell surface. Interestingly, by the presence of galactose (0.1%), the Ty21a cultures begin to lyse 2-3 hours after galactose addition. When the vaccine strain S. typhi Ty21a is grown in a medium containing limiting galactose (0.001%), the strain does not undergo lysis but builds up LPS which is a prerequisite for its immunogenicity (Kopeco et al. submitted). Therefore the hemolysin expression and secretion in different media containing 0.001% galactose were tested. It was found that the strain Ty21a carrying the plasmid pANN202-812 encoding hly operon (hly-CABD), cultured in media with 0.001% galactose, exhibited a similar amount of expressed and secreted hemolysin than the same strain cultured in corresponding media without galactose (data not shown).

### 3. In Vitro Effect of RpoS on Hemolysin Expression and Secretion:

Interestingly, an additional mutation in the rpoS gene, which also contributes to the avirulence of the Ty21a strain [4] was inherited from the wild-type parental strain Ty2. This mutation, based on the insertion of a single base, is apparently one of the reasons for the poor capacity of Ty21a to survive starvation conditions and resist various environmental stress conditions [5]. This, combined with the low shedding rate, reduces the environmental risks posed by use of Ty21a. The role of RpoS in the virulence of S. typhi is unknown.

In order to test the effect of the rpoS on the hemolysin secretion first a plasmid encoding the rpoS gene of S. typhimurium SL7207 was constructed. For this purpose, a PCR fragment encoding the whole rpoS gene with the promoter region was cloned in vector plasmid pAYC148 as described in material and methods. The functionality of resulting plasmid pRpoS in Ty21a was analysed by a stress test (material and methods). The data demonstrated that pRpoS plasmid is functional in Ty21 and that the rpoS complemented Ty21a strain is less stress sensitive than Ty21a alone. As expected, S. typhimurium SL7207 showed the best survival rate in this assay (Table 2).

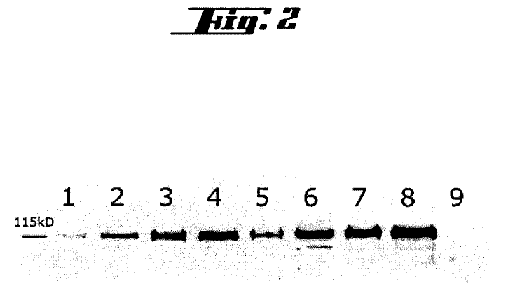

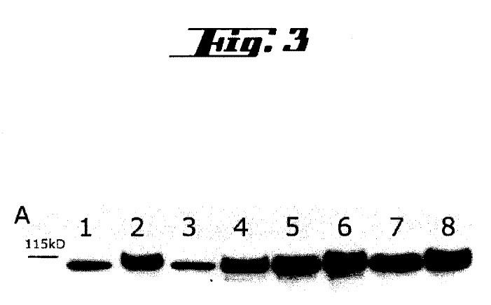

In addition, it was found that after complementation with pRpoS, the hemolysin-expressing strain Ty21a/pANN202-812 strain shows an increased expression and secretion of hemolysin compared with the same strain without rpoS plasmid (FIG. 2). One reason for this phenomenon could be the fact that RpoS is involved in the growth-dependent regulation of rfaH transcription and O antigen expression in S. typhi [19]. RfaH enhances elongation of Escherichia coli hlyCABD mRNA [20] and thus the expression and secretion of hemolysin. Therefore, the effects of rpoS and rfaH were analyzed by Real-time reverse transcription-PCR (RT-PCR) and Western blot. The first method was performed to measure the transcriptional levels of the hlyA (hemolysin), hlyD (hemolysin translocator) and rfaH genes, as well as the cat gene (plasmid antibiotic resistance) using a Rotor-gene system and SYBR green 1. The data demonstrated that the complementation with rfaH lead to a significant increase of hemolysin expression (FIG. 3A) and secretion in Ty21a, most likely by anti-termination of the polycistronic hlyCABD mRNA (FIGS. 3B and C). Interestingly, the level of rfaH mRNA is highly increased even in the early logarithmic growth phase (FIG. 3B). It was shown that transcription of rfaH in S. typhi is growth phase dependant with peak expression at the end of the logarithmic phase [21]. This tight regulation seems to be altered by introducing multiple copies of rfaH through pRfaH. The effect of rpoS on expression and secretion is not fully clear, RT-PCR analysis revealed only slight, if any upregulation of rfaH and a twofold upregulation of hlyA (not shown). RpoS could regulate HlyA secretion and expression at least in parts by mechanisms distinct from antitermination via RfaH.

4. rpoS and rfaH Effects on Invasion and Survival of S. typhi Ty21a in RAW 264.7 Macrophages:

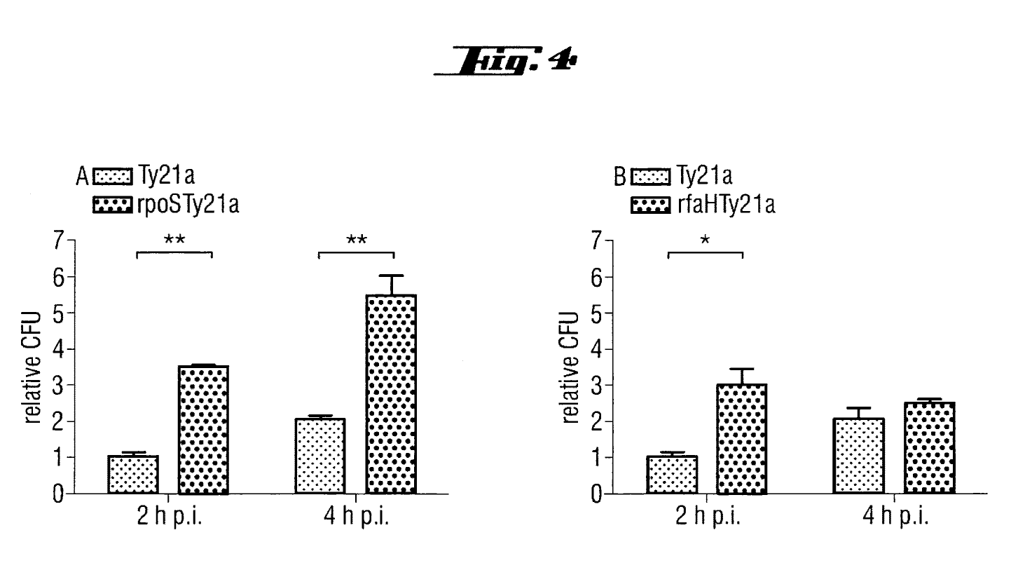

In order to test whether the serotype S. typhi Ty21a RpoS⁺ or RfaH⁺ are able to invade and survive better in RAW macrophages than Ty21a, an invasion and survival assay was performed as described in material and methods (FIG. 4). Significant differences between the CFUs of S. typhi Ty21a and S. typhi Ty21a RpoS⁺ strains were found 2 and 4 hours after infection. RfaH in contrast, only conferred a benefit during early time-points. Four hours after infection, the number of intracellular bacteria is equal for Ty21a and rfaHTy21a. While Ty21a replicates intracellularly with a doubling time of 2 hours, rfaHTy21a does not seem to show significant intracellular growth.

5. An Enhancement of Antibody Responses Against Hemolysin But Not LPS After Intranasal Immunization with S. typhi Strains Secreting HlyA:

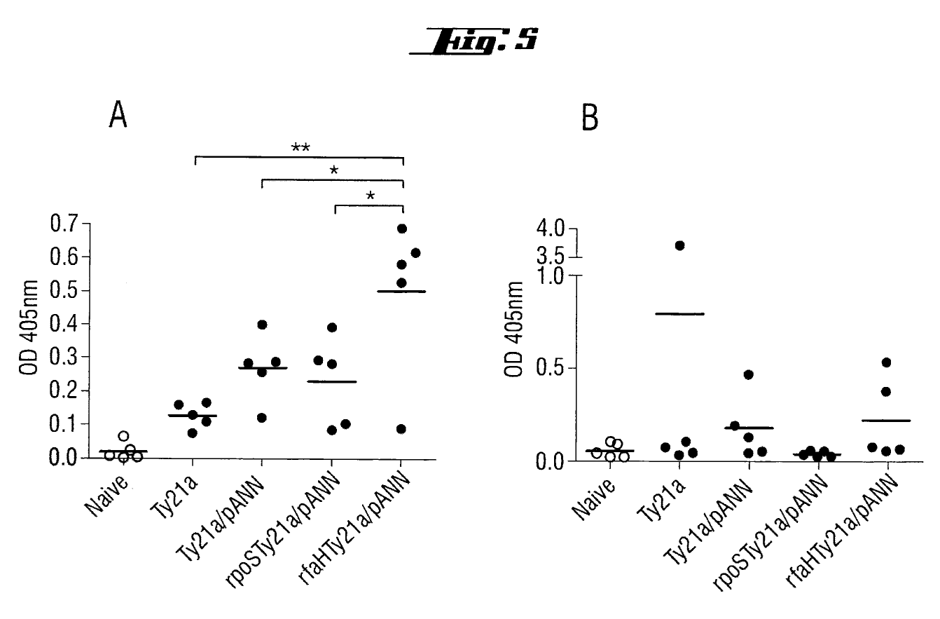

It was already demonstrated previously that Ty21a maintains the hemolysin expression vector pANN 202-812 after immunization of mice and that i.n. immunization of mice with Ty21a/pANN 202-812 results in IgG responses against the heterologous antigen HlyA [22]. In order to test the immunological effect of rpoS and rfaH, the humoral immune responses against HlyA and LPS, the immunodominant antigen of Ty21a in vivo, was also assessed. In this experiment, five groups of Balb/c mice (n=25) were immunized i.n twice with rpoSTy21a/pANN202-812, rfaHTy21a/pANN202-812, Ty21a/pANN202-812, Ty21a (control) and naïve mice. Induction of HlyA and LPS-specific immune responses was analyzed on day 49 p.i. by HlyA and LPS-specific ELISA (FIG. 5). Interestingly, immunization with the rfaHTy21a/pANN 202-81 strain revealed a significant enhancement of antibody responses against HlyA (FIG. 5A), but not LPS (FIG. 5B) in comparison to all others groups. Furthermore, the difference in Hly-specific antibody responses between experimental groups immunized with rfaHTy21a/pANN202-812 and rpoSTy21a/pANN202-812 was also statistically significant (p<0.05), as determined by 1-way ANOVA followed by Newman-Keuls multiple comparison test. The overall reactivity of the sera against LPS was rather low, only 4 of 25 mice were responding to this antigen, even though detection was carried out with anti IgG and IgM antibodies in this case.

### Discussion

The attenuated live bacterial vaccine strain S. typhi Ty21a may be a suitable carrier for heterologous proteins in human. Heterologous antigen expression in Ty21a was recently tested in two clinical trials which showed that Ty21a delivering antigens from H. pylori in its cytoplasm is safe and exhibits some immunogenicity after oral immunization with experimental formulations [6, 7]. However, several preclinical studies have shown that surface display or secretion are more suitable for heterologous antigen delivery by attenuated Salmonella strains [8, 9, 23, 24] than the cytoplasmic expression employed in the above-mentioned clinical trials [6, 7]. Therefore, recently the E. coli hemolysin secretion system was assessed for the delivery of heterologous antigens by Ty21a [22, 25]. The hemolysin secretion system was chosen because it has been used in numerous preclinical studies for the delivery of antigens from bacteria, viruses, and parasites by attenuated Salmonella strains [10], as well as in the immunotherapy of tumors [26, 27], as a delivery system for immunocontraceptive vaccines [28], and as a system for the co-expression and codelivery of active cytokines [29].

In addition, it was already demonstrated that after transformation of a hemolysin expression vector into Ty21a, HlyA was expressed and secreted successfully by the vaccine strain after growth and formulation according to procedures used for production of the licensed Vivotif® vaccine [22]. The plasmid encoding the antigen secretion system was also stably maintained in vitro as well as in vivo. In the past, cytosolic expression of heterologous antigens in attenuated Salmonella strains has been shown to pose a strong metabolic burden to the carrier bacteria [30]. This in turn reduces the in vivo persistence of the bacteria and the stability of heterologous antigen expression. However, the data suggest that due to the secretion of the heterologous antigens, both persistence of the vaccine bacteria and antigen stability are improved [22].

Here, the improvement of S. typhi Ty21a for hemolysin expression and secretion is described. It was found that after complementation with pRfaH, the hemolysin-expressing strain Ty21a/pANN202-812 strain shows a highly increased expression and secretion of hemolysin compared with the same strain without rfaH plasmid (FIG. 3A). The reason for these finding seems to be the antitermination of the polycistronic mRNA. Furthermore, rfaHTy21a/pANN202-812 induced significantly higher antibody titres than control strains and even rpoSTy21/pANN202-812 in BI/6 mice immunised i.n. (FIG. 5). This is surprising as rpoSTy21a/pANN202-812 also showed increased expression and secretion of HlyA (FIG. 2). One reason for this could be the increased survival inside macrophages as explained in the next section.

Ty21 complemented with rfaH exhibited a higher titer of intracellular bacteria within the first 2 hours of infection of macrophages compared to Ty21a alone. This benefit is lost after 4 hours because rfaHTy21a does not seem to replicate or the balance between killing and replication is shifted towards killing of the bacteria (FIG. 4B). The reason for this is not known, maybe rfaH mediates increased uptake by macrophages and/or increased susceptibility against killing by macrophages. Ty21a complemented with rpoS shows increased intracellular survival in RAW macrophages compared to the respective wildtype (FIG. 4A). The data correspond to a study by Alama et al in which a S. typhi rpoS-negative strain was more susceptible to intracellular killing by RAW macrophages. This killing was dependant on the action of nitric oxide synthetase [31]. In contrast to that, a S. typhi rpoS deletion mutant showed no such susceptibility to intracellular killing in resting THP-I cells, a human acute monocytic leukemia cell line. However, this mutant was less cytotoxic than the respective wildtype [32]. This indicates that RpoS could play a role in the virulence of serovar typhi strains. Furthermore, the observed reduced susceptibility might result in less efficient MHC presentation of major antigens, which in turn could lead to reduced immunogenicity of the RpoS-positive strain like shown for a phoP mutant of serovar typhimurium [33] Therefore, resistance against intracellular killing might explain the low antibody titres against the examined antigens (FIG. 5).

Both genes, rpoS and rfaH, contribute to virulence in Salmonella strains. Mutants of these factors were evaluated as live vaccine vectors in several studies [4, 35-38]. Introduction of these genes into the attenuated Ty21a strain might therefore increase virulence and affect attenuation. However, the contribution of rpoS in S. typhi virulence remains unclear. Furthermore, the attenuating effect of the rfaH mutation in S. typhimurium is mainly due to down-regulation of virulence factors by silencing of LPS synthesis genes [39]. In contrast, Ty21a represents a LPS defective strain resulting in a rough phenotype [1,2]. It is tempting to assume that overexpression of rfaH in Ty21a will not interfere with safety concerns as full LPS synthesis is abrogated downstream of this regulator.

The present data clearly show that rfaHTy21a, secreting heterologous antigens via T1SS, allows vaccination against both the carrier antigen and typhoid fever. Thereby, recombinant Ty21a/rfaH strains may form the basis for a novel generation of combination vaccines which can be administered orally.

TABLE 1

Bacterial strains and DNAs (Ap^{R}-ampicillin-resistant;
Cm^{R}-chloramphenicol-resistant)

| | | |
|---|---|---|
| | | Source or |
| Name | Relevant characteristics/sequence | reference |

Bacterial strains:

| | | |
|---|---|---|
| E. coil DH5 | F⁻, Φ80dlacZ M15, (lacZYA-argF)U169 | Invitrogen, |
| | deoR, recA1, endA1, hsdR17(rk⁻, mk⁺), | Karlsruhe, |
| | phoA, supE44, I⁻, thi-1, gyrA96, relA1 | Germany |
| S. enterica | S. typhi Ty2, galE, rpoS, viaB | Berna |
| serovar | | Biotech |
| Typhi Ty21a | | Ltd. |
| Salmonella | hisG46, DEL407 [aroA544::Tn10 (Tc^{S})] | Stocker, |
| enterica | | B.A.D |
| serovar | | |
| Typhimurium | | |
| aroA | | |
| SL7207 | | |
| Salmonella | aroA, fliC::Tn10 | Stocker, |
| enterica | | B.A.D |
| serovar | | |
| dublin aroA | | |
| SL5928 | | |

Oligos:

| | | |
|---|---|---|
| rpoS_up | 5′CATCGCCTGGATCCCCGGGAACG3′ | this study |
| | SEQ ID NO: 1 | |
| rpoS_down | 5′GACGCAAAAAGCTTTTGATGACGCGCC3′ | this study |
| | SEQ ID NO: 2 | |
| rfaH_up | 5′GAGGATCCACAGGAAGCTTGATGCGTTTTAG3′ | this study |
| | SEQ ID NO: 3 | |
| rfaH_down | 5′CGCAAGATTTAGGGATCCTTCAGAATACGACC3′ | this study |
| | SEQ ID NO: 4 | |
| htrB_up | 5′GCGAGAATACGGAGAATTG3′ SEQ ID NO: 5 | this study |
| htrB2_down | 5′GAGGGGAAAAATTGCAG3′ SEQ ID NO: 6 | this study |

Plasmids:

| | | |
|---|---|---|
| pANN202- | AP^{R}, hlyR, C, A, B, D, derivate of pBR322 | [40] |
| 812 | | |
| pACYC184 | Cm^{R}, Tet^{R} | [41] |
| pRpoS | Cm^{R} derivate of pACY184, encoding rpoS | this study |
| | gene of S. typhimurium SL7207 | |
| pRfaH | Cm^{R} derivate of pACY184, encoding rfaH | this study |
| | gene of S. typhi Ty21a | |

TABLE 2
Oxidative stress test

| Strain | 3 mM H₂O₂ | 30 mM H₂O₂ |
|---|---|---|
| Ty21a | 0% | 0% |
| rpoSTy21a | 2% | 0% |
| SL7207 | 15% | 0% |

Survival rate of bacterial cultures treated with H₂O₂. Cells were grown to the mid-logaritmic phase, treated with the indicated concentration H₂O₂ of and plated on BHI agar. The survival rate was determined by counting CFUs of treated and untreated cells.

TABLE 3
Primers

| | | |
|---|---|---|
| RT Primers | sequence | detected mRNA |

| | | |
|---|---|---|
| Cat RT (F) | 5′ACGTTTCAGTTTGCTCATGG | chloramphenicol transacety- |
| | 3′ SEQ ID NO:7 | lase mRNA |
| Cat RT (R) | 5′CCGGCCTTTATTCACATTCT | chloramphenicol transacety- |
| | 3′ SEQ ID NO:8 | lase mRNA |
| HlyA RT (F) | 5′CAGCTGCAGGTAGCTTCG | bicistronic mRNACA and |
| | 3′ SEQ ID NO:9 | polycistronic mRNACABD |
| HlyA RT (R) | 5′TATGCTGATGTGGTCAGGGT | bicistronic mRNACA and |
| | 3′ SEQ ID NO:10 | polycistronic mRNACABD |
| HlyD RT (F) | 5′ATTCTTACCCGCTCATCTGG | polycistronic mRNACABD |
| | 3′ SEQ ID NO:11 | |
| HlyD RT (R) | 5′GTGGCAACAATTTCCACTTG | polycistronic mRNACABD |
| | 3′ SEQ ID NO:12 | |
| RfaH RT (F) | 5′AACGTACCTTCGTCAGCGA | rfaH |
| | 3′ SEQ ID NO:13 | |
| RfaH RT (R) | 5′GTGGCGTTGATTGTAGTGGT | rfaH |
| | 3′ SEQ ID NO:14 | |

### REFERENCES

1. Germanier, R. and E. Furer, Isolation and characterization of Gal E mutant Ty 21a of Salmonella typhi: a candidate strain for a live, oral typhoid vaccine. Infect Dis, 1975. 131(5): p. 553-8.
2. Germanier, R. and E. Furer, Immunity in experimental salmonellosis. II. Basis for the avirulence and protective capacity of gal E mutants of Salmonella typhimurium. Infect Immun, 1971. 4(6): p. 663-73.
3. Fukasawa, T. and H. Nikaido, Galactose-sensitive mutants of Salmonella. II. Bacteriolysis induced by galactose. Biochim Biophys Acta, 1961. 48: p. 470-83.
4. Coynault, C., V. Robbe-Saule, and F. Norel, Virulence and vaccine potential of Salmonella typhimurium mutants deficient in the expression of the RpoS (sigma S) regulon. Mol Microbiol, 1996. 22(1): p. 149-60.
5. Robbe-Saule, V., C. Coynault, and F. Norel, The live oral typhoid vaccine Ty21a is a rpoS mutant and is susceptible to various environmental stresses. FEMS Microbiol Lett, 1995. 126(2): p. 171-6.
6. Bumann, D., et al., Safety and immunogenicity of live recombinant Salmonella enterica serovar Typhi Ty21a expressing urease A and B from Helicobacter pylori in human volunteers. Vaccine, 2001. 20(5-6): p. 845-52.
7. Metzger, W. G., et al., Impact of vector-priming on the immunogenicity of a live recombinant Salmonella enterica serovar typhi Ty21a vaccine expressing urease A and B from Helicobacter pylori in human volunteers. Vaccine, 2004. 22(17-18): p. 2273-7.
8. Hess, J., et al., Superior efficacy of secreted over somatic antigen display in recombinant Salmonella vaccine induced protection against listeriosis. Proc Natl Acad Sci USA, 1996. 93(4): p. 1458-63.
9. Russmann, H., et al., Delivery of epitopes by the Salmonella type III secretion system for vaccine development. Science, 1998. 281(5376): p. 565-8.
10. Gentschev, I., G. Dietrich, and W. Goebel, The E. coli alpha-hemolysin secretion system and its use in vaccine development. Trends Microbiol, 2002. 10(1): p. 39-45.
11. Wandersman, C. and P. Delepelaire, TolC, an Escherichia coli outer membrane protein required for hemolysin secretion. Proc Natl Acad Sci USA, 1990. 87(12): p. 4776-80.
12. Gentschev, I., et al., Development of antigen-delivery systems, based on the Escherichia coli hemolysin secretion pathway. Gene, 1996. 179(1): p. 133-40.
13. Spreng, S., et al., The Escherichia coli haemolysin secretion apparatus: a potential universal antigen delivery system in gram-negative bacterial vaccine carriers. Mol Microbiol, 1999. 31(5): p. 1596-8.
14. Robbe-Saule, V., et al., Characterization of the RpoS status of clinical isolates of Salmonella enterica. Appl Environ Microbiol, 2003. 69(8): p. 4352-8.
15. Laemmli, U.K., Cleavage of structural proteins during the assembly of the head of bacteriophage T4. Nature, 1970. 227(5259): p. 680-5.
16. Gentschev, I., et al., Mini-TnhlyAs: a new tool for the construction of secreted fusion proteins. Mol Gen Genet, 1996. 252(3): p. 266-74.
17. Westphal, O., Lüderitz, O., Bister, F, Über die Extraktion von Bakterien mit Phenol-Wasser. Z. Naturforsch., 1952. 7.
18. Warburg, O., Christian, W., Isolierung und Kristallisation des Gärungsferments Enolase. Biochem. Z., 1942. 310.
19. Bittner, M., et al., RpoS and RpoN are involved in the growth-dependent regulation of rfaH transcription and O antigen expression in Salmonella enterica serovar Typhi. Microb Pathog, 2004. 36(1): p. 19-24.
20. Leeds, J. A. and R. A. Welch, RfaH enhances elongation of Escherichia coli hlyCABD mRNA. J Bacteriol, 1996. 178(7): p. 1850-7.
21. Rojas, G., et al., The rfaH gene, which affects lipopolysaccharide synthesis in Salmonella enterica serovar Typhi, is differentially expressed during the bacterial growth phase. FEMS Microbiol Lett, 2001. 204(1): p. 123-8.
22. Gentschev, I., et al., Use of the alpha-hemolysin secretion system of Escherichia coli for antigen delivery in the Salmonella typhi Ty21a vaccine strain. Int J Med Microbiol, 2004. 294(6): p. 363-71.
23. Spreng, S., et al., Protection against murine listeriosis by oral vaccination with recombinant Salmonella expressing protective listerial epitopes within a surface-exposed loop of the TolC-protein. Vaccine, 2003. 21(7-8): p. 746-52.
24. Gentschev, I., et al., Delivery of protein antigens and DNA by attenuated intracellular bacteria. Int J Med Microbiol, 2002. 291(6-7): p. 577-82.
25. Gentschev, I., et al., Vivotif—a ‘magic shield’ for protection against typhoid fever and delivery of heterologous antigens. Chemotherapy, 2007. 53(3): p. 177-80.
26. Gentschev, I., et al., Use of a recombinant Salmonella enterica serovar Typhimurium strain expressing C-Raf for protection against C-Raf induced lung adenoma in mice. BMC Cancer, 2005. 5(1): p. 15.
27. Fensterle, J., et al., Cancer Immunotherapy based on recombinant Salmonella enterica serovar Typhimurium aroA strains secreting prostate-specific antigen (PSA) and cholera toxin subunit B (CtxB). Cancer Gene Therapy.
28. Donner, P., Goebel, W., Demuth, A., Gentschev, I., Hess, J., Kaufmann, S. H. E., Use of a secretion vector for fertility control by oral vaccination. Patent WO-09850067, 1998.
29. Hahn, H. P., et al., A Salmonella typhimurium strain genetically engineered to secrete effectively a bioactive human interleukin (hIL)-6 via the Escherichia coli hemolysin secretion apparatus. FEMS Immunol Med Microbiol, 1998. 20(2): p. 111-9.
30. Galen, J. E. and M. M. Levine, Can a ‘flawless’ live vector vaccine strain be engineered? Trends Microbiol, 2001. 9(8): p. 372-6.
31. Alam, M. S., et al., Involvement of Salmonella enterica serovar Typhi RpoS in resistance to NO-mediated host defense against serovar Typhi infection. Microbial Pathogenesis, 2006. 40(3): p. 116-125.
32. Khan, A. Q., et al., Salmonella typhi rpoS mutant is less cytotoxic than the parent strain but survives inside resting THP-1 macrophages. FEMS Microbiol Lett, 1998. 161(1): p. 201-8.
33. Wick, M. J., et al., The phoP locus influences processing and presentation of Salmonella typhimurium antigens by activated macrophages. Mol Microbiol, 1995. 16(3): p. 465-76.
34. Curtiss, R., 3rd and C. A. Nickerson, Recombinant avirulent immunogenic S typhi having rpos positive phenotype. U.S. Pat. No. 6,024,961, 2000.
35. Fang, F. C., et al., The alternative sigma factor katF (rpoS) regulates Salmonella virulence. Proc Natl Acad Sci USA, 1992. 89(24): p. 11978-82.
36. Nickerson, C. A. and R. Curtiss, 3rd, Role of sigma factor RpoS in initial stages of Salmonella typhimurium infection. Infect Immun, 1997. 65(5): p. 1814-23.
37. Lee, H. Y., et al., Evaluation of phoP and rpoS mutants of Salmonella enterica serovar Typhi as attenuated typhoid vaccine candidates: virulence and protective immune responses in intranasally immunized mice. FEMS Immunol Med Microbiol, 2007. 51(2): p. 310-8.
38. Nagy, G., et al., Oral immunization with an rfaH mutant elicits protection against salmonellosis in mice. Infect Immun, 2004. 72(7): p. 4297-301.
39. Nagy, G., et al., Down-regulation of key virulence factors makes the Salmonella enterica serovar Typhimurium rfaH mutant a promising live-attenuated vaccine candidate. Infect Immun, 2006. 74(10): p. 5914-25.
40. Vogel, M., et al., Characterization of a sequence (hlyR) which enhances synthesis and secretion of hemolysin in Escherichia coli. Mol Gen Genet, 1988. 212(1): p. 76-84.
41. Chang, A. C. and S. N. Cohen, Construction and characterization of amplifiable multicopy DNA cloning vehicles derived from the P15A cryptic miniplasmid. J Bacteriol, 1978. 134(3): p. 1141-56.

1. A recombinant bacterium, which comprises
at least one nucleotide sequence coding for a E. coli hemolysin secretion system, wherein the at least one nucleotide sequence comprises a full length or partial HlyA, HlyB and HlyD gene sequence under control of a hly-specific promoter or not a hly-specific bacterial promoter, and
at least one nucleotide sequence coding for a protein that increases expression, increases secretion or increases secretion and expression of a full length or partial HlyA compared to normal/wild-type HlyA expression and/or secretion.

2. The recombinant bacterium according to claim 1, in which an rpoS gene is deleted or inactivated.

3. The recombinant bacterium according to claim 1, which further comprises at least one nucleotide sequence comprising a rfaH gene, a rpoN gene, or a combination of these, wherein the nucleotide sequence is integrated into a bacterial chromosome.

4. The recombinant bacterium according to claim 1, which further comprises at least one nucleotide sequence comprising a rfaH gene, a rpoN gene, or a combination of these, wherein the nucleotide sequence is on a plasmid.

5. The recombinant bacterium according to claim 1, wherein the bacterium is attenuated.

6. The recombinant bacterium according to claim 5, wherein the attenuation is caused by deletion or inactivation of at least one gene selected from the group consisting of: aroA, aro, asd, gal, pur, cya, crp, phoP/Q, and omp.

7. The recombinant bacterium according to claim 5, wherein the attenuation results in an auxotrophic bacterium.

8. The recombinant bacterium according to claim 1, wherein the bacterium is a gram-negative bacterium or a gram-positive bacterium.

9. The recombinant bacterium according to claim 1, wherein the bacterium is selected from the group consisting of: Shigella spp., Salmonella spp., Listeria spp., Escherichia spp., Mycobacterium spp., Yersinia spp., Vibrio spp., and Pseudomonas spp.

10. The recombinant bacterium according to claim 9, wherein the bacterium is selected from the group consisting of: Shigella flexneri, Salmonella typhimurium, Mycobacterium bovis BCG, Listeria monocytogenes, Salmonella typhi, Yersinia enterocolitica, Vibrio cholerae, and Escherichia coli.

11. The recombinant bacterium according to claim 9, wherein the bacterium is selected from the group consisting of Salmonella typhi Ty2, or Salmonella typhi Ty21a.

12. The recombinant bacterium according to claim 1, which further comprises
at least one nucleotide sequence coding for at least one complete or partial antigen of at least one wild-type or mutated protein; and
at least one nucleotide sequence coding for at least one protein toxin and/or at least one protein toxin subunit.

13. The recombinant bacterium according to claim 12, wherein the at least one complete or partial antigen of at least one wild-type or mutated protein is selected from the group consisting of a receptor; an extracellular portion of a receptor, a transmembrane portion of a receptor, an intracellular portion of a receptor; an adhesion molecule; an extracellular portion of an adhesion molecule, a transmembrane portion of an adhesion molecule, an intracellular portion of an adhesion molecule; a signal-transducing protein; a cell-cycle protein; a transcription factor; a differentiation protein; an embryonic protein; a viral protein; an allergen; a protein of a microbial pathogen; a protein of a eukaryotic pathogen; a cancer testis antigen protein; a tumor antigen protein; glandula thyroidea specific protein, glandula mammaria specific protein, glandula salivaria specific protein, nodus lymphoideus specific protein, glandula mammaria specific protein, tunica mucosa gastris specific protein, kidney specific protein, ovarium specific protein, prostate specific protein, cervix specific protein, tunica serosa vesicae urinariae specific protein and nevus specific protein.

14. The recombinant bacterium according to 12, wherein the at least one complete or partial antigen of at least one wild-type or mutated protein is selected from the group consisting of Her-2/neu, androgen receptor, estrogen receptor, midkine receptor, EGF receptor, ERBB2, ERBB4, TRAIL receptor, FAS, TNFalpha receptor, TGF-beta receptor, lactoferrin receptor, basic myelin, alpha-lactalbumin, GFAP, fibrillary acid protein, tyrosinase, EGR-1, MUC1, c-Raf (Raf-1), A-Raf, B-Raf, B-Raf V599E, B-Raf V600E, B-Raf KD, B-Raf V600E kinase domain, B-Raf V600E KD, B-Raf V600E kinase domain KD, B-Raf kinase domain, B-Raf kinase domain KD, N-Ras, K-Ras, H-Ras, Bcl-2, Bcl-X, Bcl-W, Bfl-1, Brag-1, Mcl-1, A1, Bax, BAD, Bak, Bcl-Xs, Bid, Bik, Hrk, Bcr/abl, Myb, C-Met, IAP1, IAO2, XIAP, ML-IAP LIVIN, survivin, APAF-1, cyclin D(1-3), cyclin E, cyclin A, cyclin B, cyclin H, Cdk-1, Cdk-2, Cdk-4, Cdk-6, Cdk-7, Cdc25C, p16, p15, p21, p27, p18, pRb, p107, p130, E2F(1-5), GMD45, MDM2, PCNA, ARF, PTEN, APC, BRCA, Akt, PI3K, mTOR, p53 and homologues, C-Myc, NFkB, c-Jun, ATF-2, Sp1, prostate specific antigen (PSA), carcinoembryonic antigen, alpha-fetoprotein, PAP; PSMA; STEAP; MAGE, MAGE-1, MAGE-3, NY-ESO-1, PSCA, MART, Gp100, tyrosinase, GRP, TCF-4, viral antigens of the viruses HIV, HPV, HCV, HPV, EBV, CMV, HSV, influenza virus, influenza virus type A, influenza virus type A (H5N1) and (H3N2), influenza virus type B, influenza virus type C; hemagglutinins, hemagglutinin H1, hemagglutinin H5, hemagglutinin H7, hemagglutinin HA1, hemagglutinin HA12, hemagglutinin HA12C, neuramidase, p60, LLO, urease, CSP, calflagin and CPB.

15. The recombinant bacterium according to 12, wherein the at least one complete or partial antigen of at least one wild-type or mutated protein is selected from the group consisting of is a kinase selected from the group of consisting of AAK1 (NM 014911), MTK (NM 004920), ABL1 (NM 005157), ABL2 (NM 005158), ACK1 (NM 005781), ACVR1 (NM 001105), ACVR1B (NM 020328), ACVR2 (NM 001616), ACVR2B (NM 001106), ACVRL1 (NM 000020), ADCK1 (NM 020421), ADCK2 (NM 052853), ADCK4 (NM 024876), ADCK5 (NM 174922), ADRBK1 (NM 001619), ADRBK2 (NM 005160), AKT1 (NM 005163), AKT2 (NM 001626), AKT3 (NM 005465), ALK (NM 004304), ALK7 (NM 145259), ALS2CR2 (NM 018571), ALS2CR7 (NM 139158), AMHR2 (NM 020547), ANKK1 (NM 178510), ANKRD3 (NM 020639), APEG1 (NM 005876), ARAF (NM 001654), ARK5 (NM 014840), ATM (NM 000051), ATR (NM 001184), AURKA (NM 003600), AURKB (NM 004217), AURKC (NM 003160), AXL (NM 001699), BCKDK (NM 005881), BCR (NM 004327), BIKE (NM 017593), BLK (NM 001715), BMPR1A (NM 004329), BMPR1B (NM 001203), BMPR2 (NM 001204), BMX (NM 001721), BRAF (NM 004333), BRD2 (NM 005104), BRD3 (NM 007371), BRD4 (NM 014299), BRDT (NM 001726), BRSK1 (NM 032430), BRSK2 (NM 003957), BTK (NM 000061), BUB1 (NM 004336), BUB1B (NM 001211), CABC1 (NM 020247), CAMK1 (NM 003656), CaMK1b (NM 198452), CAMK1D (NM 020397), CAMK1G (NM 020439), CAMK2A (NM 015981), CAMK2B (NM 001220), CAMK2D (NM 001221), CAMK2G (NM 001222), CAMK4 (NM 001744), CAMKK1 (NM 032294), CAMKK2 (NM 006549), CASK (NM 003688), CCRK (NM 012119), CDC2 (NM 001786), CDC2L1 (NM 001787), CDC2L5 (NM 003718), CDC42BPA (NM 014826), CDC42BPB (NM 006035), CDC7L1 (NM 003503), CDK10 (NM 003674), CDK11 (NM 015076), CDK2 (NM 001798), CDK3 (NM 001258), CDK4 (NM 000075), CDK5 (NM 004935), CDK6 (NM 001259), CDK7 (NM 001799), CDK8 (NM 001260), CDK9 (NM 001261), CDKL1 (NM 004196), CDKL2 (NM 003948), CDKL3 (NM 016508), CDKL4 (NM 001009565), CDKL5 (NM 003159), CHEK1 (NM 001274), CHUK (NM 001278), CIT (NM 007174), CLK1 (NM 004071), CLK2 (NM 003993), CLK3 (NM 003992), CLK4 (NM 020666), CRK7 (NM 016507), CSF1R (NM 005211), CSK (NM 004383), CSNK1A1 (NM 001892), CSNK1D (NM 001893), CSNK1E (NM 001894), CSNK1G1 (NM 022048), CSNK1G2 (NM 001319), CSNK1G3 (NM 004384), CSNK2A1 (NM 001895), CSNK2A2 (NM 001896), DAPK1 (NM 004938), DAPK2 (NM 014326), DAPK3 (NM 001348), DCAMKL1 (NM 004734), DCAMKL2 (NM 152619), DCAMKL3 (XM 047355), DDR1 (NM 013993), DDR2 (NM 006182), DMPK (NM 004409), DMPK2 (NM 017525.1), DYRK1A (NM 001396), DYRK1B (NM 006484), DYRK2 (NM 006482), DYRK3 (NM 003582), DYRK4 (NM 003845), EEF2K (NM 013302), EGFR (NM 005228), EIF2AK3 (NM 004836), EIF2AK4 (NM 001013703), EPHA1 (NM 005232), EPHA10 (NM 001004338), EPHA2 (NM 004431), EPHA3 (NM 005233), EPHA4 (NM 004438), EPHA5 (NM 004439), EPHA6 (XM 114973), EPHA7 (NM 004440), EPHA8 (NM 020526), EPHB1 (NM 004441), EPHB2 (NM 017449), EPHB3 (NM 004443), EPHB4 (NM 004444), EPHB6 (NM 004445), ERBB2 (NM 004448), ERBB3 (NM 001982), ERBB4 (NM 005235), ERK8 (NM 139021), ERN1 (NM 001433), ERN2 (NM 033266), FASTK (NM 025096), FER (NM 005246), FES (NM 002005), FGFR1 (NM 000604), FGFR2 (NM 022970), FGFR3 (NM 000142), FGFR4 (NM 022963), FGR (NM 005248), FLJ23074 (NM 025052), FLJ23119 (NM 024652), FLJ23356 (NM 032237), FLT1 (NM 002019), FLT3 (NM 004119), FLT4 (NM 002020), FRAP1 (NM 004958), FRK (NM 002031), FYN (NM 002037), GAK (NM 005255), GPRK5 (NM 005308), GPRK6 (NM 002082), GPRK7 (NM 139209), GRK4 (NM 005307), GSG2 (NM 031965), GSK3A (NM 019884), GSK3B (NM 002093), GUCY2C (NM 004963), GUCY2D (NM 000180), GUCY2F (NM 001522), H11 (NM 014365), HAK (NM 052947), HCK (NM 002110), HIPK1 (NM 152696), HIPK2 (NM 022740), HIPK3 (NM 005734), HIPK4 (NM 144685), HR1 (NM 014413), HUNK (NM 014586), ICK (NM 016513), IGF1R (NM 000875), IKBKB (NM 001556), IKBKE (NM 014002), ILK (NM 004517), INSR (NM 000208), INSRR (NM 014215), IRAK1 (NM 001569), IRAK2 (NM 001570), IRAK3 (NM 007199), IRAK4 (NM 016123), ITK (NM 005546), JAK1 (NM 002227), JAK2 (NM 004972), JAK3 (NM 000215), KDR (NM 002253), KIS (NM 144624), KIT (NM 000222), KSR (XM 290793), KSR2 (NM 173598), LAK (NM 025144), LATS1 (NM 004690), LATS2 (NM 014572), LCK (NM 005356), LIMK1 (NM 016735), LIMK2 (NM 005569), LMR3 (XM 055866), LMTK2 (NM 014916), LOC149420 (NM 152835), LOC51086 (NM 015978), LRRK2 (XM 058513), LTK (NM 002344), LYN (NM 002350), MAK (NM 005906), MAP2K1 (NM 002755), MAP2K2 (NM 030662), MAP2K3 (NM 002756), MAP2K4 (NM 003010), MAP2K5 (NM 002757), MAP2K6 (NM 002758), MAP2K7 (NM 005043), MAP3K1 (XM 042066), MAP3K10 (NM 002446), MAP3K11 (NM 002419), MAP3K12 (NM 006301), MAP3K13 (NM 004721), MAP3K14 (NM 003954), MAP3K2 (NM 006609), MAP3K3 (NM 002401), MAP3K4 (NM 005922), MAP3K5 (NM 005923), MAP3K6 (NM 004672), MAP3K7 (NM 003188), MAP3K8 (NM 005204), MAP3K9 (NM 033141), MAP4K1 (NM 007181), MAP4K2 (NM 004579), MAP4K3 (NM 003618), MAP4K4 (NM 145686), MAP4K5 (NM 006575), MAPK1 (NM 002745), MAPK10 (NM 002753), MAPK11 (NM 002751), MAPK12 (NM 002969), MAPK13 (NM 002754), MAPK14 (NM 001315), MAPK3 (NM 002746), MAPK4 (NM 002747), MAPK6 (NM 002748), MAPK7 (NM 002749), MAPK8 (NM 002750), MAPK9 (NM 002752), MAPKAPK2 (NM 032960), MAPKAPK3 (NM 004635), MAPKAPK5 (NM 003668), MARK (NM 018650), MARK2 (NM 017490), MARK3 (NM 002376), MARK4 (NM 031417), MAST1 (NM 014975), MAST205 (NM 015112), MAST3 (XM 038150), MAST4 (XM 291141), MASTL (NM 032844), MATK (NM 139355), MELK (NM 014791), MERTK (NM 006343), MET (NM 000245), MGC33182 (NM 145203), MGC42105 (NM 153361), MGC43306 (C9orf96), MGC8407 (NM 024046), MIDORI (NM 020778), MINK (NM 015716), MKNK1 (NM 003684), MKNK2 (NM 017572), MLCK (NM 182493), MLK4 (NM 032435), MLKL (NM 152649), MOS (NM 005372), MST1R (NM 002447), MST4 (NM 016542), MUSK (NM 005592), MYLK (NM 053025), MYLK2 (NM 033118), MYO3A (NM 017433), MYO3B (NM 138995), NEK1 (NM 012224), NEK10 (NM 152534), NEK11 (NM 024800), NEK2 (NM 002497), NEK3 (NM 002498), NEK4 (NM 003157), NEK5 (MGC75495), NEK6 (NM 014397), NEK7 (NM 133494), NEK8 (NM 178170), NEK9 (NM 033116), NLK (NM 016231), NPR1 (NM 000906), NPR2 (NM 003995), NRBP (NM 013392), NRBP2 (NM 178564), NRK (NM 198465), NTRK1 (NM 002529), NTRK2 (NM 006180), NTRK3 (NM 002530), OBSCN (NM 052843), OSR1 (NM 005109), PACE-1 (NM 020423), PAK1 (NM 002576), PAK2 (NM 002577), PAK3 (NM 002578), PAK-4 (NM 005884), PAK6 (NM 020168), PAK7 (NM 020341), PASK (NM 015148), PCTK1 (NM 006201), PCTK2 (NM 002595), PCTK3 (NM 212503), PDGFRA (NM 006206), PDGFRB (NM 002609), PDK1 (NM 002610), PDK2 (NM 002611), PDK3 (NM 005391), PDK4 (NM 002612), PDPK1 (NM 002613), PFTK1 (NM 012395), PHKG1 (NM 006213), PHKG2 (NM 000294), PIK3R4 (NM 014602), PIM1 (NM 002648), PIM2 (NM 006875), PIM3 (NM 001001852), PINK1 (NM 032409), PKE (NM 173575), PKMYT1 (NM 004203), pknbeta (NM 013355), PLK (NM 005030), PLK3 (NM 004073), PRKAA1 (NM 006251), PRKAA2 (NM 006252), PRKACA (NM 002730), PRKACB (NM 002731), PRKACG (NM 002732), PRKCA (NM 002737), PRKCB1 (NM 002738), PRKCD (NM 006254), PRKCE (NM 005400), PRKCG (NM 002739), PRKCH (NM 006255), PRKCI (NM 002740), PRKCL1 (NM 002741), PRKCL2 (NM 006256), PRKCM (NM 002742), PRKCN (NM 005813), PRKCQ (NM 006257), PRKCZ (NM 002744), PRKD2 (NM 016457), PRKDC (NM 006904), PRKG1 (NM 006258), PRKG2 (NM 006259), PRKR (NM 002759), PRKWNK1 (NM 018979), PRKWNK2 (NM 006648), PRKWNK3 (NM 020922), PRKWNK4 (NM 032387), PRKX (NM 005044), PRKY (NM 002760), PRPF4B (NM 003913), PSKH1 (NM 006742), PSKH2 (NM 033126), PTK2 (NM 005607), PTK2B (NM 004103), PTK6 (NM 005975), PTK7 (NM 002821), PTK9 (NM 002822), PTK9L (NM 007284), PXK (NM 017771), QSK (NM 025164), RAD53 (NM 007194), RAF1 (NM 002880), RAGE (NM 014226), RET (NM 020975), RHOK (NM 002929), RIOK1 (NM 031480), RIOK2 (NM 018343), RIPK1 (NM 003804), RIPK2 (NM 003821), RIPK3 (NM 006871), RIPK5 (NM 015375), RNASEL (NM 021133), ROCK1 (NM 005406), ROCK2 (NM 004850), ROR1 (NM 005012), ROR2 (NM 004560), ROS1 (NM 002944), RPS6KA1 (NM 002953), RPS6KA2 (NM 021135), RPS6KA3 (NM 004586), RPS6KA4 (NM 003942), RPS6KA5 (NM 004755), RPS6KA6 (NM 014496), RPS6 KB1 (NM 003161), RPS6 KB2 (NM 003952), RPS6KC1 (NM 012424), RPS6KL1 (NM 031464), RYK (NM 002958), SBK (XM 370948), SCYL1 (NM 020680), SCYL2 (NM 017988), SGK (NM 005627), SgK069 (SU SgK069), SgK085 (XM 373109), SgK110 (SU SgK110), SGK2 (NM 016276), SgK223 (XM 291277), SgK269 (XM 370878), SgK424 (CGP SgK424), SgK493 (SU_SgK493), SgK494 (NM 144610), SgK495 (NM 032017), SGKL (NM 013257), SK681 (NM 001001671), SLK (NM 014720), SMG1 (NM 015092), SNARK (NM 030952), SNF1LK (NM 173354), SNF1LK2 (NM 015191), SNK (NM 006622), SNRK (NM 017719), SRC (NM 005417), SRMS (NM 080823), SRPK1 (NM 003137), SRPK2 (NM 003138), SSTK (NM 032037), STK10 (NM 005990), STK11 (NM 000455), STK16 (NM 003691), STK17A (NM 004760), STK17B (NM 004226), STK18 (NM 014264), STK19 (NM 032454), STK22B (NM 053006), STK22C (NM 052841), STK22D (NM 032028), STK23 (NM 014370), STK24 (NM 003576), STK25 (NM 006374), STK3 (NM 006281), STK31 (NM 031414), STK32B (NM 018401), STK33 (NM 030906), STK35 (NM 080836), STK36 (NM 015690), STK38 (NM 007271), STK38L (NM 015000), STK39 (NM 013233), STK4 (NM 006282), STLK5 (NM 001003787), STYK1 (NM 018423), SUDD (NM 003831), SYK (NM 003177), TAF1 (NM 138923), TAF1L (NM 153809), TAO1 (NM 004783), TAOK1 (NM 020791), TAOK3 (NM 016281), TBCK (NM 033115), TBK1 (NM 013254), TEC (NM 003215), TEK (NM 000459), TESK1 (NM 006285), TESK2 (NM 007170), TEX14 (NM 031272), TGFBR1 (NM 004612), TGFBR2 (NM 003242), TIE (NM 005424), TIF1 (NM 003852), TLK1 (NM 012290), TLK2 (NM 006852), TNIK (NM 015028), TNK1 (NM 003985), TOPK (NM 018492), TP53RK (NM 033550), TRAD (NM 007064), TRIB1 (NM 025195), TRIB2 (NM 021643), TRIB3 (NM 021158), TRIM28 (NM 005762), TRIM33 (NM 015906), TRIO (NM 007118), TRPM6 (NM 017662), TRPM7 (NM 017672), TRRAP (NM 003496), TSSK4 (NM 174944), TTBK1 (NM 032538), TTBK2 (NM 173500), TTK (NM 003318), TTN (NM 003319), TXK (NM 003328), TYK2 (NM 003331), TYRO3 (NM 006293), ULK1 (NM 003565), ULK2 (NM 014683), ULK3 (NM 015518), ULK4 (NM 017886), VRK1 (NM 003384), VRK2 (NM 006296), VRK3 (NM 016440), WEE1 (NM 003390), Wee1B (NM 173677), YANK1 (NM 145001), YES1 (NM 005433), ZAK (NM 016653), and ZAP70 (NM 001079).

16. The recombinant bacterium according to claim 12, wherein the at least one protein toxin and/or at least one protein toxin subunit is selected from the group consisting of a bacterial toxin, an enterotoxin, an exotoxin, a type I toxin, a type II toxin, a type III toxin, a type IV toxin, a type V toxin, a RTX toxin, an AB toxin, an A-B toxin, an A/B toxin, an A+B toxin, an A-5B toxin and an AB5 toxin.

17. The recombinant bacterium according to claim 16, wherein the at least one protein toxin and/or at least one protein toxin subunit is selected from the group consisting of Adenylate cyclase toxin, Anthrax toxin, Anthrax toxin (EF), Anthrax toxin (LF), Botulinum toxin, Cholera toxin (CT, Ctx), Cholera toxin subunit B (CTB, CtxB), Diphtheria toxin (DT, Dtx), E. coli LT toxin, E. coli heat labile enterotoxin (LT), E. coli heat labile enterotoxin subunit B (LTB), E. coli ST toxin, E. coli heat stabile enterotoxin (ST), Erythrogenic toxin, Exfoliatin toxin, Exotoxin A, Perfringens enterotoxin, Pertussis toxin (PT, Ptx), Shiga toxin (ST, Stx), Shiga toxin subunit B (STB, StxB), Shiga-like toxin, Staphylococcus enterotoxins, Tetanus toxin (TT), Toxic shock syndrome toxin (TSST-1), Vero toxin (VT), Toxin A (TA) and Toxin B (TB) of Clostridium difficile, Lethal Toxin (LT) and Hemorrhagic Toxin (HT) of Clostridium sordellii, and alpha Toxin (AT) of Clostridium novyi.

18. The recombinant bacterium according to claim 12, wherein the at least one complete or partial antigen of at least one wild-type or mutated protein and the at least one protein toxin and/or at least one protein toxin subunit are linked together expressed and/or secreted as a fusion protein.

19. The recombinant bacterium according to claim 18, wherein the fusion protein is selected from the group consisting of CtxB-PSA, CtxB-B-Raf V600E KD, CtxB-B-Raf V600E kinase domain, CtxB-B-Raf V600E kinase domain KD, CtxB-B-Raf, CtxB-B-Raf KD, CtxB B-Raf kinase domain KD, CtxB-HA1, and CtxB-HA12C.

20. A process for producing the recombinant bacterium according to claim 1, comprising
(a) transforming a bacterium with at least one nucleotide sequence coding for an E. coli hemolysin secretion system, wherein the at least one nucleotide sequence comprises a full length or partial HlyA, HlyB and HlyD gene sequence under control of a hly-specific promoter or not hly-specific bacterial promoter, wherein the at least one nucleotide sequence is integrated into a bacterial chromosome or on a plasmid,
(b) complementing the bacterium of a) with at least one nucleotide sequence coding for a protein that increases expression, secretion or both expression and secretion of a full length or partial HlyA compared to normal/wild-type HlyA expression and/or secretion, and optionally comprising rfaH and/or rpoN gene integrated into the bacterial chromosome or on a plasmid
(c) optionally, deleting or inactivating an rpoS gene in the bacterium of b)
(d) optionally, attenuating the bacterium of b) or c), optionally by deleting or inactivating at least one gene selected from the group consisting of aroA, aro, asd, gal, pur, cya, crp, phoP/Q, and omp.
(e) optionally, transforming the bacterium of b), c), or d) with at least one nucleotide sequence coding for at least one complete or partial antigen of at least one wild-type or mutated protein and at least one nucleotide sequence coding for at least one protein toxin and/or at least one protein toxin subunit, and which is integrated into the bacterial chromosome or on a plasmid.

21. A pharmaceutical composition comprising at least one recombinant bacterium according to claim 1, and a pharmaceutically acceptable carrier.

22. The pharmaceutical composition according to claim 21, wherein the at least one recombinant bacterium is lyophilized.

23. The pharmaceutical composition according to claim 21, wherein the pharmaceutically acceptable carrier is a capsule.

24. A method for treating a physiological and/or pathophysiological condition selected from the group consisting of a disease involving macrophage inflammations where macrophages are associated with disease onset or disease progression, a tumor disease, uncontrolled cell division, a malignant tumor, a benign tumor, a solid tumor, a sarcoma, a carcinoma, a hyperproliferative disorder, a carcinoid, Ewing sarcoma, Kaposi sarcoma, a brain tumor, a tumor originating from a brain, a tumor originating from a nervous system, a tumor originating from a meninge, a glioma, a neuroblastoma, stomach cancer, kidney cancer, a kidney cell carcinoma, prostate cancer, a prostate carcinoma, a connective tissue tumor, a soft tissue sarcoma, a pancreatic tumor, a liver tumor, a head tumor, a neck tumor, esophageal cancer, thyroid cancer, osteosarcoma, retinoblastoma, thymoma, testicular cancer, lung cancer, bronchial carcinoma, breast cancer, mamma carcinoma, intestinal cancer, colorectal tumor, colon carcinoma, rectal carcinoma, a gynecological tumor, an ovarian tumor, uterine cancer, cervical cancer, cervix carcinoma, cancer of a body of a uterus, corpus carcinoma, endometrial carcinoma, urinary bladder cancer, bladder cancer, skin cancer, basalioma, spinalioma, melanoma, intraocular melanoma, leukemia, chronic leukemia, acute leukemia, lymphoma, infection, viral infection, bacterial infection, influenza, chronic inflammation, organ rejection, an autoimmune disease, diabetes and diabetes type II, the method comprising
administering an effective amount at least one recombinant bacterium according to claim 1 to an individual in need thereof.

25. A pharmaceutical kit comprising at least one recombinant bacterium according to claim 1; and a pharmacologically acceptable buffer.
